(19) United States
(12) Patent Application Publication
(10) Pub. No.: US 2005/0192758 A1
(43) Pub. Date: Sep. 1, 2005

(54) Methods for comparing functional sites in proteins

(76) Inventor: Lei Xie, San Diego, CA (US)

Correspondence Address: PERKINS COIE LLP, POST OFFICE BOX 1208, SEATTLE, WA, 98111-1208, US

(21) Appl. No.: 10/917,959

(22) Filed: Aug. 13, 2004

Related U.S. Application Data
(60) Provisional application No. 60/495,652, filed on Aug. 15, 2003.

(51) Int. Cl. G06F 19/00; G01N 33/48; G01N 33/50

(52) U.S. Cl. 702/19

(57) ABSTRACT

The present invention relates to methods and systems for representing and scoring the similarity of two protein by iteratively rotating and translating one protein surface representation relative to the other protein surface representation in order to maximize (or minimize) a score that represents both the volume between the two surface representations and the similarity in the identities and positions of the residues comprising the two protein surfaces. In another aspect of the invention, such methods and systems are used to compare and annotate a protein comprising a putative functional site of unknown function with a database of reference proteins of known function.

### CROSS-REFERENCE TO RELATED APPLICATIONS

This application claims the benefit of U.S. Provisional Application No. 60/495,652 filed Aug. 15, 2003, which is incorporated by reference as if contained herein in its entirety.

### STATEMENT REGARDING FEDERALLY SPONSORED RESEARCH OR DEVELOPMENT

Not Applicable

### REFERENCE TO A COMPUTER PROGRAM APPENDIX

Not Applicable

### BACKGROUND OF THE INVENTION

Protein surfaces often contain biologically functional sites such as catalytic sites, ligand binding sites, protein-protein recognition sites and protein anchoring sites. The identification and characterization (referred to as annotation) of functional sites allows for the identification of new biochemical pathways and protein mediated interactions, and also supplements the body of science relating to known pathways and systems. More importantly, functional site annotation may also be used for target identification and validation, to rationalize small molecule screening and to guide medicinal chemistry efforts once a small molecule has been successfully screened against a potential drug target.

Current methods for functionally annotating a putative functional site are based upon comparing the primary sequence conservation, topography, and physiochemical properties between a putative functional site and one or more annotated functional sites.

Primary sequence conservation based methods compare a putative functional site on the surface of a protein of interest, referred to throughout as a query protein, to one or more annotated functional sites on the surface of one or more comparison proteins, referred to throughout as reference proteins, by determining, whether and to what extent, the putative functional site comprises residues which are evolutionally conserved across the annotated functional sites. Needleman, S. B., Wunsch, C. B., A General Method Applicable to the Search for Similarities in the Amino-Acid Sequence Of Two Proteins, J. Mol. Biol., 48, 443-453 (1970); Waterman, M. S., General Methods for Sequence Comparison, Bull. Math. Biol. 46, 473-500 (1984); Landgraf, R., Xenarios, I., Eisenberg, D., Three Dimensional Cluster Analysis Identifies Interfaces And Functional Residues In Proteins, J. Mol Biol 307(5):1487-502 (2001). While in many cases the residues that comprise functional sites are highly conserved across functionally related proteins, this is not always the case. In order to compare evolutionarily distant proteins, other sequence independent comparison methods, such as structure based or physiochemical comparison based methods are required.

Since structural similarity is often conserved even at very low sequence homologies, structure comparison methods may be used for functional site annotation when primary sequence methods fail. Structure comparison methods may be classified as three-dimensional comparison methods or two-dimensional protein surface comparison methods. Three dimensional structure comparison methods, as exemplified in CATH, SCOP and Dali assignments, are useful for making gross functional annotations but they are of limited value for characterizing functional residues on the surface of a protein. Protein surface comparison methods are inherently harder to implement than sequence or three dimensional comparison methods since they are generally much more computationally intensive and require accurate three dimensional structures.

A number of approaches have been advanced for comparing protein surfaces. Functional motif based approaches represent a functional cluster as a set of residues and corresponding distance constraints. A database of known functional reference motifs is compared against a query structure to identify the presence of any such functional motifs in the query structure. Functional motifs may be represented and compared using graph methods. Mitchell, E. M., Artymiuk, P. J., Use of Techniques Derived From Graph Theory To Compare Secondary Structure Motifs in Proteins, 243 J. Mol. Biol. 327-344 (1994).

An alternative approach defines a molecular skin or surface volume formed by adding a predefined offset or ‘skin depth’ to the solvent accessible surface of a protein. Two protein surfaces are compared by maximizing the overlap volume of their respective skins by rigid transformations. Masek, B. B., Merchant, A. and Matthew, B., Molecular Skins: A New Concept for Quantitative Shape Matching of a Protein with its Small Molecule Mimics, Proteins Struct. Funct. Genet. 17: 193-202 (1993).

These earlier protein surface comparison methods only consider the contours—i.e. the topography—of a protein's surface. They do not consider the identities and positions of the amino-acid residues that form a protein's surface—i.e. the physiochemical topography of a protein. As used herein, the physiochemical topography of a protein refers to the three-dimensional distribution of physiochemical interaction sites on the surface of a protein. A physiochemical interaction site, also referred to herein as a physiochemical center, is a site on or near the surface of a protein that may form chemical interactions, such as hydrogen bonds, hydrophobic interactions, van der Waals interactions, or ionic interactions, with a protein binding ligand. Depending upon the level of detail regarding a protein's surface, a physiochemical interaction site may mean a cluster of physiochemically similar residues, a particular surface residue, or a particular functional group that comprises a particular residue side chain.

Klebe et. al have recently introduced a method for comparing the physiochemical topography of two protein surfaces or sub-surfaces. Schmitt, S., Kuhn D., and Klebe, G., A New Method to Detect Related Function Among Proteins Independent of Sequence and Fold Homology, J. Mol. Biol. 323, 387-406 (2002). In Klebe's method, the topography of a functional cluster of residues is described by a set of pseudocenters. Klebe uses five types of pseudocenters: hydrogen bond donor, hydrogen bond acceptor, mixed donor acceptor, hydrophobic aliphatic and hydrophobic aromatic. Each type of pseudocenter is assigned to the one or more chemical functional groups—e.g. amino groups, hydroxyl groups, aliphatic groups—that comprise each residue side chain. Two functional clusters are then compared by determining the similarity of their two graph representations using clique detection algorithms.

Klebe's method, like the geometric hashing and other graph based methods detailed immediately above, compare the similarity of two protein surfaces or sub-surfaces by discretizing the surfaces and minimizing the distances between discrete points on the respective surfaces.

The present invention generally relates to improved methods for comparing the physiochemical topography of two protein surfaces and their application for functionally annotating a protein or a functional site on the surface of a protein. The claimed protein surface comparison methods differ from the current point-distance techniques detailed above significantly. Whereas, the current methods, Klebe's included, compare the topography (or physiochemical topography) of two functional sites by minimizing the distance between discrete points, the claimed methods compare two protein surfaces by minimizing the volume between two mathematical surfaces that represent the respective topographies of the proteins being compared. This surface comparison scheme offers the prospective advantage that when one surface may be characterized as a symmetry subset of a second surface—i.e. a half sphere and a sphere, the claimed surface comparison methods should be more accurate than point-distance based approaches.

The claimed functional site comparison methods differ from the ‘molecular skins’ approach in at least three important ways. One, the ‘molecular skins’ approach only considers the topography of the functional site; it does not consider the physiochemical similarity of the two sites. Two, the molecular skins approach constructs a pseudo-volume to represent the topography of a protein surface. Three, the similarity of the two surfaces is scored by determining the maximum overlap volume that may be found by iteratively translating and rotating the pseudo-volumes. By contrast, the claimed methods score the similarity of two functional sites by determining the minimum volume that may be found between the two mathematical surfaces that represent the two sites by iteratively translating and rotating the surfaces. To demonstrate the computational efficiencies of the claimed methods over the molecular skins approach, assume that a surface may be represented by b² points, and its corresponding ‘molecular skin’ may be represented by b²a points. Thus, for each rigid translation or rotation, the claimed methods require an order of O(a) less calculations.

Another aspect of the improved surface comparison methods according to the invention is a method for representing a functional site as a plurality of connected spherical caps. This representation allows for a computationally efficient scheme for comparing the physiochemical topography of two functional sites.

### BRIEF DESCRIPTION OF THE FIGURES

FIG. 1 illustrates a method according to the invention for scoring the similarity of two functional sites

FIG. 2 illustrates a method according to the invention for determining a mathematical surface that represents a functional site.

FIGS. 3a-e illustrate the relationships between a functional site, its Voronoi diagram, its Delaunay tessellation, its Alpha Shape and the set of fused circles that may be drawn from the foregoing.

FIG. 4 illustrates an exemplary surface determined according to the methods illustrated in FIG. 2.

FIG. 5 illustrates a method according to the invention for determining a volume score, V, between two surfaces determined according to the methods illustrated in FIG. 2.

FIG. 6 illustrates the geometric parameters of a spherical cap.

FIG. 7 illustrates the excluded volume between two spherical caps.

FIG. 8 illustrates a method for determining the excluded volume between two spherical caps.

FIGS. 9a-f illustrate the application of the methods illustrated in FIG. 5.

FIG. 10 illustrates a method according to the invention for determining a chemical similarity score E for two surfaces determined according to the methods illustrated in FIG. 2.

FIG. 11 illustrates one method according to the invention for determining a protein similarity score between two functional sites.

FIG. 12 illustrates another method according to the invention for determining a protein similarity score between two functional sites.

FIG. 13 illustrates a method for building a hash table.

FIG. 14 illustrates one coordinate system that may be employed by methods according to the invention.

FIG. 15 illustrates a method according to the invention for determining the similarity of a functional site on a query protein with a database reference proteins.

FIG. 16 illustrates a method according to the invention for functionally annotating a query protein comprising a functional site with a database comprising a plurality of reference proteins that each comprises at least one characterized functional site.

FIG. 17 illustrates the architecture of a system according to the invention.

### SUMMARY OF THE INVENTION

The methods according to the invention relate to improved methods for representing functional sites and scoring their similarity. However, the claimed methods are not limited to only functional sites. In general, the claimed methods may be equally applied to representing and scoring the similarity of protein surfaces without regard to whether the surfaces are connected to any biological function. As used herein, a protein surface refers to the solvent accessible portion of a protein.

One aspect of the invention provides a method for representing a functional site as a surface formed from a plurality of connected spherical caps. This aspect of the invention identifies a binding site with the surface formed by the overlap of: i) a first set of spheres defined about the residues that line the binding site; and ii) the set of connected spheres inscribed by the tetrahedrons formed from a Delaunay tessellation of the binding site. The claimed surface representation methods allow analytical methods for determining the volume between two such surfaces and accordingly, efficient calculation of the physiochemical similarity scores according to the invention.

Another aspect according to the invention is an analytical method for determining the volume between two surfaces, each comprising a plurality of connected spherical caps. Because this method is very computationally efficient it enables the efficient similarity scoring of a functional site on a query protein against a large database of reference proteins.

Another aspect according to the invention is a method for determining a physiochemical similarity score S between two functional sites, represented as two mathematical surfaces that depends upon: i) the similarity in the identities and positions of the residues that comprise the two functional sites and ii) the volume between the two surfaces.

Another aspect of the invention provides a method for determining a protein similarity score for scoring the similarity of two functional sites comprising the steps of: 1) representing the structures of the first and second functional sites with respectively, a first mathematical surface and a second mathematical surface; 2) selecting an initial relative orientation between said first surface and said second surface; 3) determining a physiochemical similarity score S that depends upon: i) the similarity in the identities and positions of the residues of the two sites and ii) the volume between the two surfaces; 4) maximizing, or alternatively minimizing, the physiochemical similarity score by iteratively translating and rotating the first surface (or structure) relative to the second surface (or structure); and 5) identifying the protein similarity score with the maximized, or minimized, physiochemical score S.

The claimed methods for scoring the similarity of two functional sites may use any scheme for sampling the relative orientations that may be formed between the two surfaces that represent the two functional sites. One aspect of the invention uses graph based methods to determine the optimal overlay between the two functional sites being compared. As used herein, the optimal overlay between the first and second functional sites refers to the relative orientation between two functional site structures that minimizes the RMS (root means square) distance between overlaying residues when the two functional site structures are overlayed. One method in accordance with this aspect of the invention comprises the steps of: 1) representing the first and second functional site structures with graphs; 2) determining the optimal overlay between the two functional site structures; 3) representing the optimally overlayed first and second functional site structures with corresponding first and second surfaces; and 4) identifying a protein similarity score with the physiochemical similarity score S based upon the relative orientation of the two surfaces determined in step 3).

A still further aspect of the claimed invention is a method of annotating a query functional site with a database of annotated functional sites comprising the steps of: 1) using the functional site comparison methods according to the invention to score the similarity of the query functional site with each annotated functional site; and 2) selecting the highest scoring annotated functional site to annotate the query functional site.

A still further aspect of the invention is a computer or networked server comprising programming to perform the methods according to the invention.

### DETAILED DESCRIPTION OF THE INVENTION

Methods According to the Invention

The functional site comparison methods according to the invention are based in-part upon the realization that the more topographically similar are two surfaces, the smaller the volume is between the two surfaces. As used herein, a surface refers to a mathematical representation of a protein surface. In the limit of identical surfaces, the volume between the two surfaces is zero for at least one orientation and the surfaces are perfectly superimposed. The claimed methods are also based on the realization that a still more sensitive measure of the similarity of two functional sites would consider the similarity in the positions and identities of the residues—the physiochemical topography, in addition to the gross topography. As used herein a residue refers to an amino acid. Accordingly, the functional site comparison methods according to the invention use a similarity score that jointly depends upon both 1) the volume between two surface representations of the respective functional sites being compared; and 2) the similarity in the positions and identites of the residues that comprise the two functional sites or their corresponding surfaces.

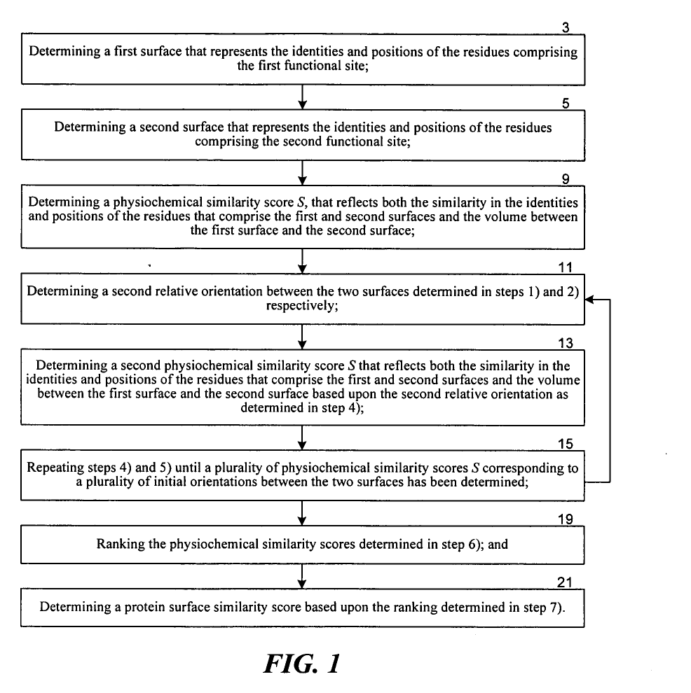

One aspect of the invention, illustrated in FIG. 1, is a method for determining a protein similarity score that reflects the similarity of a first functional site to a second functional site comprising the steps of: 1) determining a first mathematical surface that represents the identities and positions of the residues that comprise the first functional site 3; 2) determining a second mathematical surface that represents the identities and positions of the residues that comprise the second functional site 5; 3) determining a first physiochemical similarity score S that reflects both: i) the similarity of the positions and identities of the residues that comprise the two surfaces, and the ii) the volume between the two surfaces 9; 4) determining a second orientation between the two surfaces determined in steps 1) 3 and 2) 5 respectively 11; 5) determining a second physiochemical similarity score S that reflects both: i) the similarity of the positions and identities of the residues that comprise the two surfaces, and the ii) the volume between the two surfaces based upon the second orientation as determined in step 4) 13; 6) repeating steps 4) and 5) until a plurality of physiochemical similarity scores S corresponding to a plurality of relative orientations between the two surfaces have been determined 15; 7) ranking the physiochemical similarity scores determined in step 6) 19; and 8) determining a protein similarity score based upon the ranking determined in step 7) 21. As used herein, a functional site refers to a solvent accessible site on the surface of a protein that is related to the biological function of the protein. Exemplary functional sites include small molecule binding sites, catalytic sites, protein-protein interaction sites, or protein-nucleic acid binding sites. Functional site structures may be determined experimentally through co-crystallization techniques or through computational methods such as Eidogen, Inc.'s (Pasadena, Calif.) SITESEEKER algorithm. A functional site structure refers to the three dimensional arrangement of the residues that comprise a functional site.

Methods for Determining a Mathematical Surface that Represents the Physiochemical Topography of a Functional Site-3, 5

The claimed surface comparison methods may use either analytic or numeric representations of a functional site. There is no inherent limitation on how a mathematical surface is generated. Protein surface representations may be generated from the Connolly surface of a protein, with analytic splines, or from three dimensional grids. Commercially available software, such as Accelerys Inc.'s, (San Diego, Calif.) Insight II software suite, may be used to determine the Connolly surface of a protein. Grid methods represent the surface of a protein within the framework of a three-dimensional lattice of cells. One grid method represents the surface of a protein with a plurality of points and corresponding normal vectors. Via, A., Ferre, F., Brannetti, B., Helmer-Citterich, M., Protein Surface Similarities: A Survey of Methods to Describe and Compare Protein Surfaces, Cell. Mol. Life Sci. 57: 1979-1977 (2000). Each point may be further characterized or “colored” based upon the identity of the particular surface residue that the point corresponds to. Alternatively, each point may be further characterized by the physiochemical characteristics—e.g. hydrogen bond donor, hydrogen bond acceptor, hydrophobic center—of the particular residue that point represents. The shell of points and vectors is then superimposed within a lattice of cubic cells. Each point is then represented by its corresponding cubic face.

Graph Based Methods for Representing a Functional Site

A functional site may be represented as a graph where the nodes of the graph correspond to residues and the edges correspond to distances between the residues. As used herein, a residue refers to an amino acid. The methods according the invention use one or more physiochemical pseudocenters to represent each residue or groups of residues on the surface of a protein. As used herein, a pseudocenter refers to a discrete mathematical representation of a physiochemical center on the surface of a protein. A pseudocenter is characterized by: i) its type; and ii) its location. Pseudocenters may represent any level of physiochemical structure. They may represent physiochemically similar residue clusters—e.g. a hydrophobic patch, physiochemically similar residues, or physiochemically similar chemical functional groups that comprise residues. As used herein, a chemical functional group refers to a chemical functional moiety—e.g. hydroxyl groups, amino groups, or carboxylic acid groups. In one scheme there is a unique type of pseudocenter for each naturally occurring amino acid. Alternatively, the naturally occurring amino acids may be grouped into physiochemical classes, for example, hydrogen bond donors, hydrogen bond acceptors, or hydrophobic centers and a unique type of pseudocenter is assigned to each physiochemical class of residues. A still further method, advanced by Klebe, assigns pseudocenter type according to the physiochemical similarity of the functional groups that comprise residue side chains. Klebe's method, illustrated in Table 1, divides residue functional groups into five classes of pseudocenters: hydrophobic aliphatic, hydrophobic aromatic, hydrogen-bond donor, hydrogen-bond acceptor, and mixed donor-receptor. A hydrogen bond donor and a hydrogen bond acceptor refer to the two functional groups that participate in a hydrogen bond. A mixed-donor receptor refers to a functional group that can be either a hydrogen bond donor or a hydrogen bond acceptor. A hydrophobic aliphatic group refers to a functional group comprising an aliphatic hydrocarbon chain. A hydrophobic aromatic group refers to a functional group comprising an aromatic ring.

TABLE 1

| Amino | | |
|---|---|---|
| Acid | Pseudocenter Types | Pseudocenter Positions |
| Ala | Hydrophobic Aliphatic | CB |
| Arg | Hydrophobic Aliphatic | CM(CB, CG, CD) |
| | H-bond Donor | NE |
| Asn | H-bond Donor | OD1 |
| | H-bond Acceptor | ND2 |
| Asp | H-bond Donor | OD1 |
| | H-bond Acceptor | OD2 |
| Cys | Hydrophobic Aliphatic | CM(CB, SG) |
| Gln | H-bond Donor | OE1 |
| | H-bond Acceptor | NE2 |
| Glu | H-bond Acceptor | OE1 |
| | H-bond Acceptor | OE2 |
| His | Hydrophobic Aromatic | CM(CG, ND1, CD2, CE1, NE2) |
| | Mixed Donor-Acceptor | NE1 |
| | Mixed Donor-Acceptor | NE2 |
| Ile | Hydrophobic Aliphatic | CM(CB, CG1, CG2, CD1) |
| Leu | Hydrophobic Aliphatic | CM(CG, CG, CD1, CD2) |
| Lys | Hydrophobic Aliphatic | CM(CB, CG, CD, CE) |
| | H-bond Donor | NZ |
| Met | Hydrophobic Aliphatic | CM(CB, CG, SD, CE) |
| Phe | Hydrophobic Aromatic | CM(CG, CD1, CD2, CE1, CE2, CZ) |
| Pro | Hydrophobic Aliphatic | CM(CB, CG, CD) |
| Ser | Mixed Donor-Acceptor | OG |
| Thr | Hydrophobic Aliphatic | CD2 |
| | Mixed Donor-Acceptor | OD1 |
| Trp | Hydrophobic Aromatic | CM(CG, CD1, CD2, NE1, CE2, |
| | | CE3), |
| | H-bond Donor | CM(CZ1, CZ3, CH, NE1) |
| Tyr | Hydrophobic Aromatic | CM(CB, CD1, CD2, CE1, CE2, CZ), |
| | Mixed Donor-Acceptor | OH |
| Val | Hydrophobic Aliphatic | CM(CB, CG1, CG2) |
| Peptide | H-bond Acceptor | O |
| Bond | H-bond Donor | N |
| | Hydrophobic Aromatic | C |

C—Carbon,
N—Nitrogen,
S—Sulfur,
O—Oxygen,
B—Beta,
G—Gamma,
D—Delta,
E—Epsilon,
H-Eta,
Z—Zeta,
CM—Center-of-Mass

The location of each pseudocenter may be identified with the center-of-mass of the corresponding residue, side chain or functional group which that pseudocenter represents. Alternatively, a pseudocenter may be located within the van der Waals' volume of each amino acid, side chain or functional group. Chothia, C., J. Mol. Biol., 105, 1-14 (1975)

Spherical Cap Representation of a Functional Site

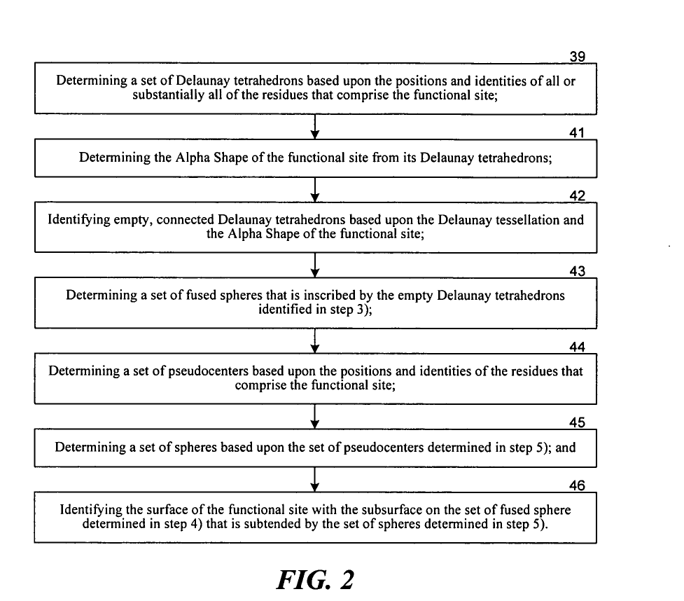

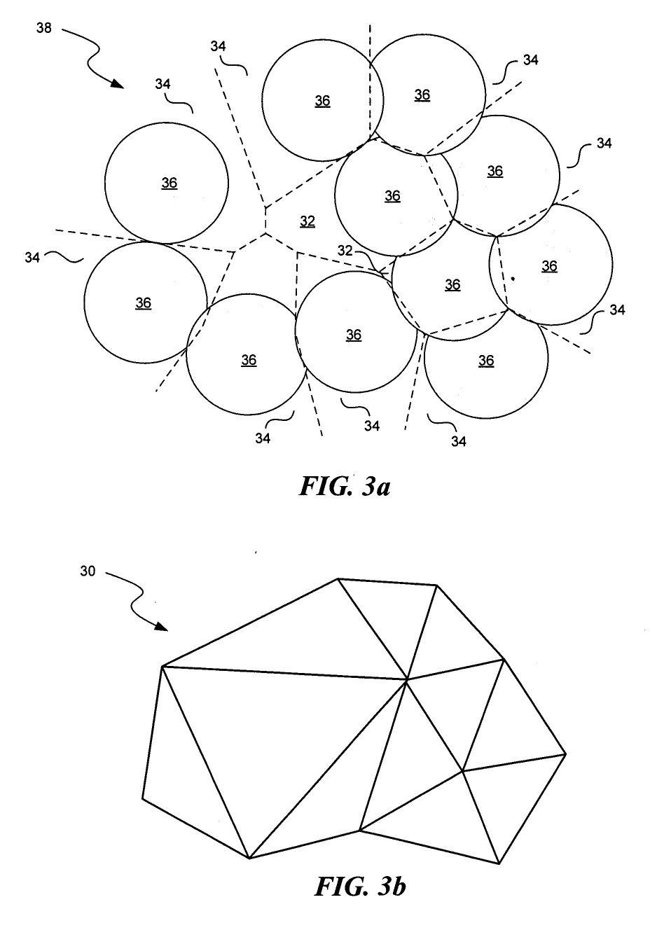

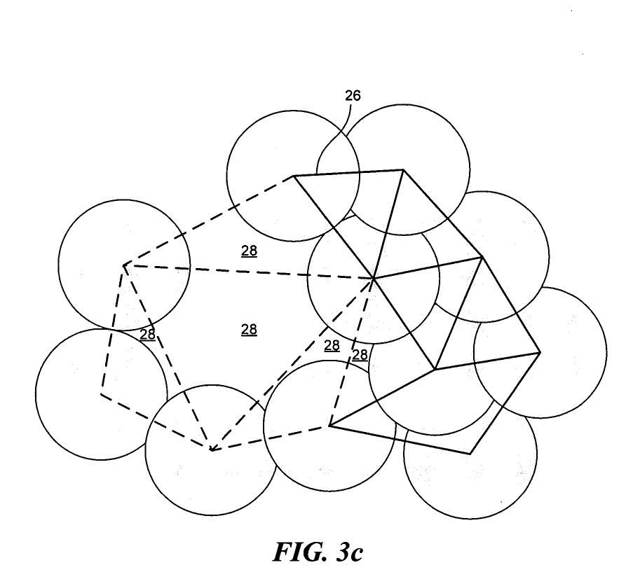

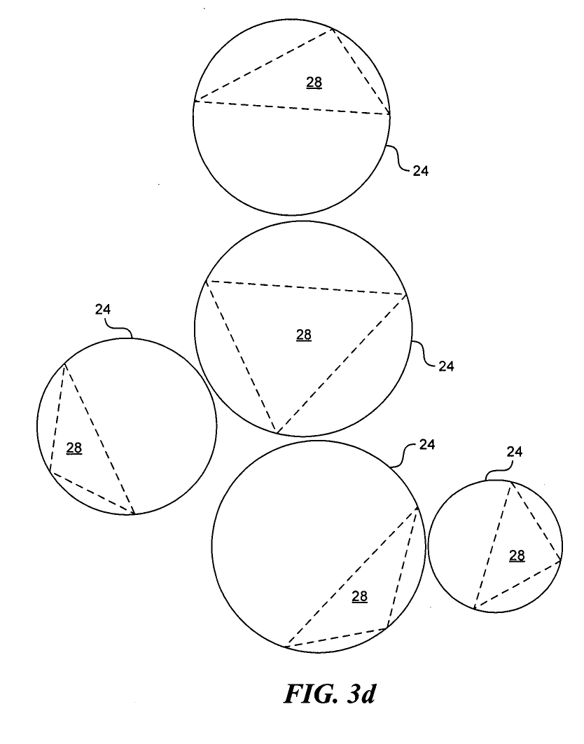

Another aspect of the invention, illustrated in FIG. 2, is a method that represents the surface of a functional site as a plurality of spherical caps comprising the steps of: 1) determining a set of Delaunay tetrahedrons based upon the positions and identities of all or substantially all of the residues that comprise the functional site 39; 2) determining the Alpha Shape of the functional site from its Delaunay tetrahedrons 41; 3) identifying empty, connected Delaunay tetrahedrons based upon the Delaunay tessellation and the Alpha Shape of the functional site 42; 4) determining a set of fused spheres that is inscribed by the empty Delaunay tetrahedrons identified in step 3) 43; 5) determining a set of pseudocenters based upon the positions and identities of the residues that comprise the functional site 44; 6) determining a set of spheres based upon the set of pseudocenters determined in step 5) 45; and 7) identifying the surface of the functional site with the subsurface on the set of fused sphere determined in step 4) 43 that is subtended by the set of spheres determined in step 5) 46. Thus, the method of FIG. 3 represents a functional site structure with the set of spherical caps {σₐ|a=1 . . . a′}, where σₐ is the spherical cap associated with a pseudocenter a.

A set of Delaunay tetrahedrons may be calculated 39 by performing a Delaunay tessellation based upon the positions of the residues that comprise a functional site and optionally, their corresponding van der Waals radii. Tables of van der Waals radii are readily available. If the functional site is on the surface of a protein found in the Protein Data Bank, the atomic radii may be assigned using the utility program PDB2ALF which is available for download at the world wide web site alphashapes.org/alpha/pdb2alf. The weighted Delaunay tessellation 39 and Alpha Shape 41 computations may be performed using the programs DELCX and MKALF, respectively. Both are also available for download at the world wide web site alphashapes.org/alpha/. DELCX calculates the Delaunay tessellation based upon the positions of all residues in a protein structure. Accordingly, these programs should be modified to determine the Delaunay tessellation of a functional site. Such modifications are well within the capacity of one ordinarily skilled in the art. The Delaunay tessellation may optionally be determined subject to the further condition that the volume of each tetrahedron corresponds to a solvent accessible volume in order to assure that only the solvent accessible surface of a functional site is considered.

FIGS. 3a-e illustrate the relationship between a functional site, its Delaunay tessellation, its Alpha Shape and the determination of a set of fused spheres. A set of fused spheres refers to a set of overlapping spheres. The Delaunay triangulation is mathematically equivalent to, and may be derived from, the Voronoi diagram of a residue cluster. FIG. 3a illustrates a radial cross-section of an exemplary functional site comprising 11 atoms and its corresponding Voronoi diagram 38. It will be appreciated by one ordinarily skilled in the art that this exemplary site comprising 11 atoms is intended for illustrative purposes only. Actual functional sites will comprise far more than 11 atoms. The Voronoi diagram comprises a plurality of Voronoi cells. Each Voronoi cell contains one atom 36. The area of each cell is defined such that the distance of each point within a particular Voronoi cell is closer to the atom of that cell than any other atom. Accordingly, two types of Voronoi cells may be identified in a Voronoi diagram: 1) open sided polygons for those boundary atoms that form the convex hull 34; and 2) closed polygons 32. FIG. 3b illustrates the Delaunay tessellation 30 corresponding to the Voronoi diagram 38 illustrated in FIG. 3a. It is formed by drawing a segment across every Voronoi edge that separates two Voronoi cells and connecting the respective atom centers of the two cells. FIG. 3c illustrates the Alpha Shape 26 corresponding to the Delauany tessellation 30 illustrated in FIG. 3b. It is formed by subtracting those triangles 28 from the Delaunay tessellation that correspond to Voronoi edges or vertices outside of the functional cluster. Such triangles are referred to as ‘empty’ Delaunay triangles 28. FIG. 3c shows these omitted edges and corresponding empty triangles with dashed lines and the corresponding Alpha Shape 26 with bold, solid lines. Referring to FIGS. 3d and 3e, since three non-collinear points uniquely define a circle (while 4 non-coplanar points uniquely define a sphere), each of the five empty Delaunay triangles 28 uniquely defines a circle 24. Accordingly, when the five empty Delaunay triangles are connected, a set five fused circles 22 is uniquely defined. While this exemplary site is illustrated in two dimensions, it will be appreciated that in three dimensions, the Delaunay triangles correspond to Delaunay tetrahedrons, the circles inscribed by the Delaunay triangles correspond to spheres and the set of fused circles corresponds to a set of fused spheres.

A set of fused spheres may be determined 43 from a set of empty Delaunay tetrahedrons by defining a sphere about the vertices of each Delaunay tetrahedron. More particularly, each sphere may be defined by reference to the four vertices of each empty Delaunay tetrahedron that inscribes the sphere. The determination of a set of fused spheres from a set of empty Delaunay tetrahedrons may optionally be subject to the further condition that the radius of each sphere must be less than approximately 20 Angstroms and be more than approximately 1.4 Angstroms. The lower limit of approximately 1.4 Angstroms is chosen to reflect the van der Waal's radius of a water molecule. The upper limit is chosen to eliminate non-physical spheres generated from the large Delaunay tetrahedrons that are sometimes generated in the Delaunay tessellation step at the ‘mouth’ of functional sites. In order to define the sub-surface on the set of fused spheres that corresponds to the functional site, a second set of spheres defined about a plurality of pseudocenters is intersected with the set of fused spheres.

A set of physiochemical pseudocenters may be determined 44 based upon the position and the identity of each or substantially each residue that comprises a functional site using the methods for determining pseudocenters detailed in the section entitled, Graph Based Methods for Representing a Protein Surface.

A set of spheres may be determined 45 from the set of pseudocenters by defining a sphere about each or substantially each pseudocenter with a radius of approximately 2.0 Angstroms to approximately 4.0 Angstroms, with approximately 2.9-3.1 Angstroms preferred. These limits are selected based upon the Lennard-Jones equilibrium distances for the typical atoms found in small molecule-protein interactions.

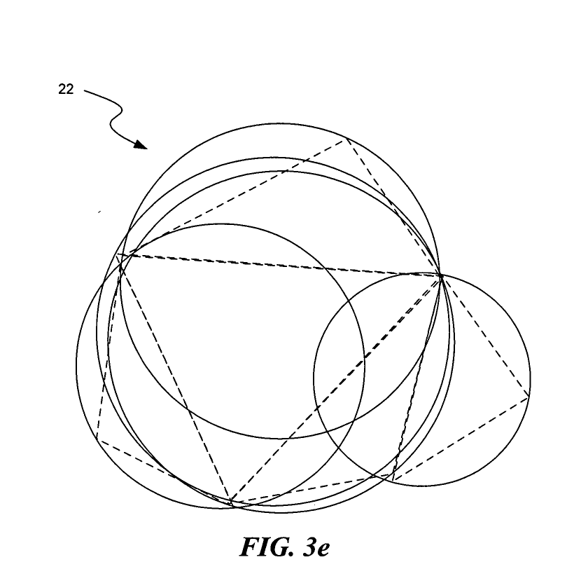

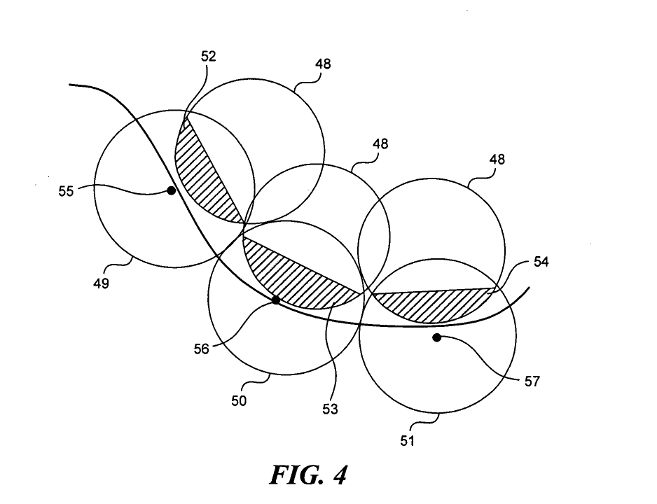

The surface of a functional site may be identified with the subsurface on the set of fused spheres determined in step 2) that is subtended by the set of spheres determined in step 4) 46. FIG. 4 illustrates the case where the first set of fused spheres 48, determined according to step 2) 41 comprises three spheres 46 and the second set of spheres determined according to step 4) 45 also comprises three spheres 49, 50, 51. The subsurface on the set of fused spheres comprises three spherical caps, 52, 53, 54 each shown by a different cross hatched region in FIG. 4.

FIG. 4 also shows that each spherical cap may be uniquely identified with one pseudocenter. More particularly, a pseudocenter corresponding to a spherical cap is the pseudocenter that is the origin for the sphere that defines a spherical cap on the surface of the set of fused spheres. In FIG. 4, pseudocenter 55 is the origin for the sphere 49 that defines spherical cap 52 on the first set of fused spheres. Similarly, pseudocenter 56 corresponds to spherical cap 53 and pseudocenter 57 corresponds to spherical cap 54. Accordingly, the physiochemical type of a particular pseudocenter may be directly associated with its corresponding spherical cap(s).

In order to identify the solvent accessible side of a surface that represents a functional site, the methods illustrated in FIG. 2 may define a unit vector normal to a surface at its midpoint pointing in the direction of the solvent. A mid point may be selected based upon the range of dimensions of a particular surface along three basis vectors defining the coordinate system.

Methods for Determining a Physiochemical Similarity Score, S-9

The functional site comparison methods according to the invention use a physiochemical similarity score S that depends upon two other scores: 1) a volume score V, that represents the volume between two mathematical surfaces, and 2) a chemical similarity score E, that reflects the similarity in the identity and positions of the residues that comprise the two surfaces.

The functional site comparison methods according to the invention use either the maximum or minimum of S as a metric for the comparison of two functional sites. Accordingly, both E and V should either be monotonically increasing or monotonically decreasing as a function of increasing similarity between the two functional sites. If E and V are not so behaved, either E or V should be rescaled so that it behaves the same as the other. To wit, if the maximum of S is being used as a similarity metric and E increases, while V decreases, with the increasing similarity of two functional sites, V should be rescaled so it increases with the increasing similarity between the functional sites. Conversely, if the minimum of S is being used as a similarity metric, E should be rescaled so that it decreases with increasing similarity between the functional sites.

If the maximum of S is used as a similarity metric and V decreases with the increasing similarity of two functional sites, V may be rescaled according to any function ƒ(V) that asymptotically behaves according to

$${lim}_{V \to 0} f ( V ) = C and {lim}_{V \to ∞} f ( V ) = 0.$$

Suitable functions include, a Half Gaussian function, a Half-Lorentzian function, or any other function that is peaked about zero or an offset and asymptotically and monotonically approaches positive zero. See mathworld.wolfram.com/GaussianFunction.html. One method rescales the volume score V, according to

$$f ( V ) = \frac{1}{β ( 1 + {ⅇ}^{γ V} )}$$

where β and γ are scaling factors.

If the minimum of S is used as a similarity metric and E increases with the increasing similarity of two functional sites, E, may be rescaled according to any monotonic function ƒ(E) that asymptotically behaves the same as ƒ(V), namely,

$${lim}_{E \to ∞} f ( E ) = 0 and {lim}_{E \to 0} f ( E ) = C .$$

Methods for Determining a Volume score V Between Two Surfaces

The claimed functional site comparison methods may use analytical or numerical methods for determining a score that represents the volume between two surfaces. Suitable numerical methods include grid based methods. Grid based methods, which are well known in the art, divide a volume into finite volume elements and sum those elements contained within a particular three-dimensional space to determine its volume. One disadvantage to numerical methods is their computational overhead relative to analytical methods.

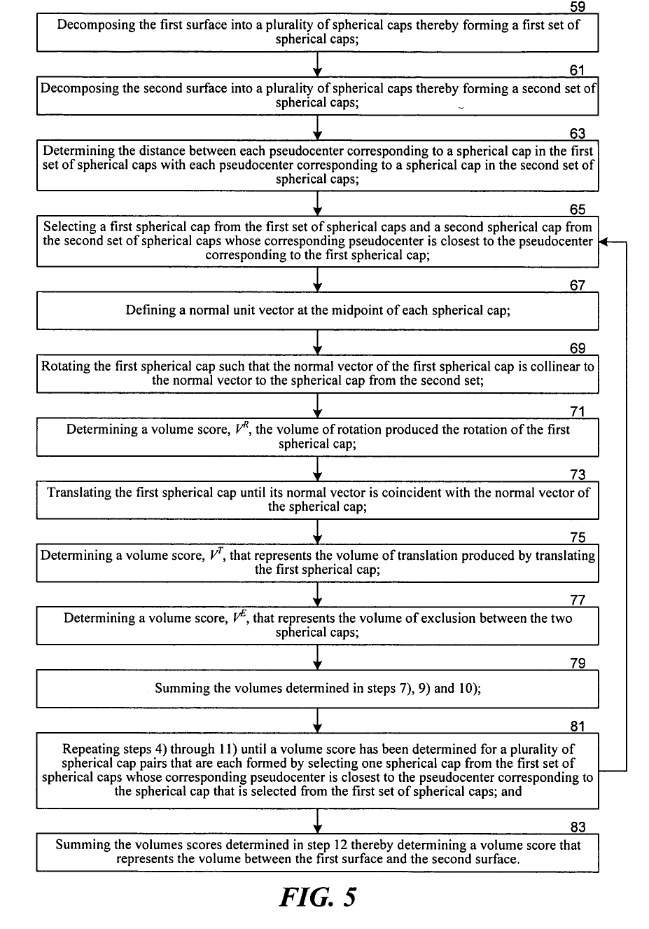

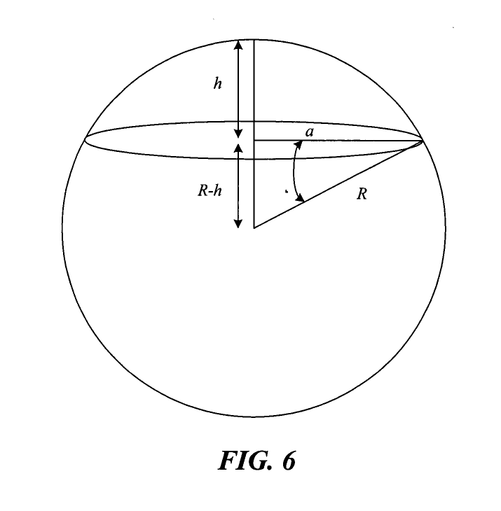

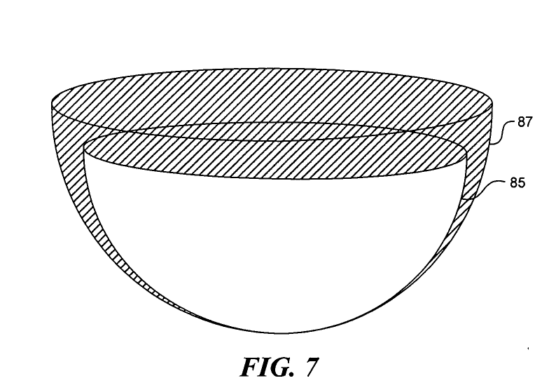

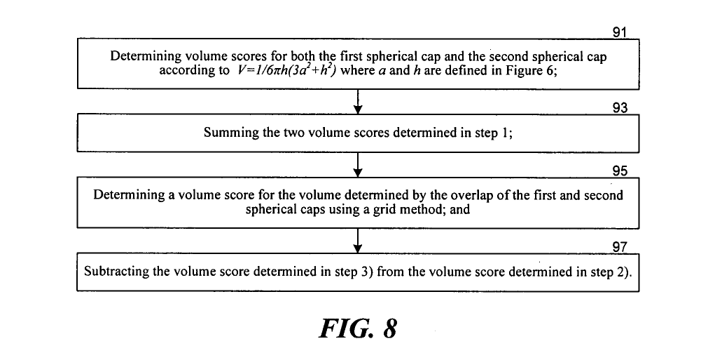

Another aspect of the invention is a computationally efficient, semi-analytical method, illustrated in FIG. 5, for determining a volume score between two surfaces determined using the methods illustrated in FIG. 2, comprising the steps of: 1) decomposing the first surface into a plurality of spherical caps thereby forming a first set of spherical caps 59; 2) decomposing the second surface into a plurality of spherical caps thereby forming a set of spherical caps 61; 3) determining the distance between each pseudocenter corresponding to a spherical cap in the first set of spherical caps and each pseudocenter corresponding to a spherical cap in the second set of spherical caps 63; 4) selecting a first spherical cap from the first set of spherical caps and a second spherical cap from the second set of spherical caps whose corresponding pseudocenter is closest to the pseudocenter corresponding to the first spherical cap 65; 5) defining a normal unit vector at the midpoint of each spherical cap 67; 6) rotating the first spherical cap such that the vector normal to the first spherical cap is collinear to the vector normal to second spherical cap 69; 7) determining a volume score V^{R} that represents the volume of rotation produced by the rotation of the first spherical cap 71; 8) translating the first spherical cap until its normal vector is coincident with the normal vector to the second spherical cap 73; 9) determining a volume score V^{T} that represents the volume produced by translating the first spherical cap 75; 10) determining a volume score V^{E} that represents the volume of exclusion between the two spherical caps 77; 11) summing the volumes determined in steps 7), 9) and 10), thereby determining a volume score that represents the volume between the spherical cap pair formed from the first and second spherical caps 79; 12) repeating steps 4) through 11) until a volume score has been determined for a plurality of spherical cap pairs that are each formed by selecting one spherical cap from the first set of spherical caps and the spherical cap from the second set of spherical caps whose corresponding pseudocenter is closest to the pseudocenter corresponding to the spherical cap that is selected from the first set of spherical caps 81; and 13) summing the volume scores determined in step 12 thereby determining a volume score that represents the volume between the first surface and the second surface 83. One ordinarily skilled in the art will appreciate that this method does not calculate the true volume between the two surfaces. Rather, this method calculates a quantity which scales with the true volume between the surfaces. This method takes advantage of the relative computational ease of determining the volume and other relevant geometric parameters associated with a spherical cap. The volume of a spherical cap may be determined according to V_{cap}=⅙πh(3a²+h²), where a and h are defined according to FIG. 6.

The first and second steps 59 61, for determining a volume score between two surfaces determined according to the methods illustrated in FIG. 2, decompose the first and second surfaces into all or substantially all of their constituent spherical caps. As used herein, decompose means identify the individual spherical caps from the plurality of spherical caps that comprise the surface. It does not imply changing their positions. Accordingly, if the first surface comprises i spherical caps and the second surface comprises j spherical caps, the first and second set of spherical caps may be denoted by Σ₁={σᵢ|i=1 . . . m} and Σ₂={σⱼ|j=1 . . . n} respectively. Since, the volume scoring methods detailed in FIG. 5 determine the volume between two surfaces by summing the volume between spherical cap pairs, the accuracy of these methods increases as the number of spherical cap pairs that are utilized increases. Still, if less accuracy is required for a particular comparison, it is not essential that the volume between each spherical cap pair be determined. Accordingly, this step recites that the volume is determined for a plurality of spherical cap pairs

A next step 63, determines the distance between each pseudocenter corresponding to a spherical cap in the first set of spherical caps and each pseudocenter corresponding to a spherical cap in the second set of spherical caps.

A next step 65, selects a first spherical cap, σᵢ₌₁, from the first set of spherical caps Σ₁, and a second spherical cap, σⱼ₌₁, from the second set of spherical caps Σ₂ whose corresponding pseudocenter is closest to the pseudocenter corresponding to the first spherical cap.

A next step 67, determines a unit normal vector to both the first and second spherical caps at their respective midpoints pointing in the direction of the solvent. In one embodiment, the unit vector originates at the midpoint on the surface of a spherical cap. But, as one skilled in the art will appreciate, the unit vector may also ‘pierce’ the spherical cap.

A next step 69, rotates the first spherical cap relative to the second spherical cap until the normal vector of the first spherical cap is collinear to the normal vector of the second spherical cap.

A next step 71, determines a score that represents the volume of rotation, Vᵢ₌₁^{R}=V₁^{R}, caused by the rotation of the first spherical cap. As used herein, a volume of rotation refers to the volume swept by the rotation of an element of finite volume, such as a spherical cap, about an axis of rotation. There is no limitation on how V₁^{R} may be determined. One convenient method that may be suitably employed determines the volume of rotation, V₁^{R}, according to V₁^{R}=θ/2ππa₁², where aᵢ₌₁ is defined in FIG. 6 and θ is the angle between the vector normal to the first spherical cap and the vector normal to the second spherical cap.

A next step 73, translates, the first spherical cap until the vector normal to it is coincident to the normal vector normal of the second spherical cap.

A next step 75, determines a volume score that represents the translational volume, Vᵢ₌₁^{T}=V₁^{T}, swept by the first spherical cap, as it is translated in step 8) 73. As used herein, the volume of translation refers to the volume swept by the translation of an element of finite volume, such as a spherical cap. There is no inherent limitation on how the volume of translation may be determined. One method that may be suitably employed determines the volume of translation, V₁^{T}, according to V₁^{T}=πa₁²d, where a₁ is defined in FIG. 5 and d is the distance the first spherical cap is translated in step 3).

A next step 77, determines a volume score that represents the excluded volume, V_{i=1,j=1}^{E}=V_{1,1}^{E}, between the first and second spherical caps. FIG. 7 illustrates the excluded volume, V_{1,1}^{E}, shown by the cross-hatched region, between two exemplary spherical caps 85, 87. The excluded volume between two spherical caps refers to the total solvent accessible volume that is unique to only one spherical cap when two spherical caps overlap. In other words, the excluded volume is the volume that is not shared by the partially overlapped spherical caps. There is no inherent limitation on how such an excluded volume may be determined. One method, illustrated in FIG. 8, that is computationally efficient since it relies in part on analytical determinations of spherical cap volume, comprises the steps of: 1) determining the volume scores of both the first spherical cap and the second spherical cap according to V_{cap}=1/6πh(3a²+h²) where a and h are defined in FIG. 6 91; 2) summing the two volume scores determined in step 1) 93; 3) determining a volume score for the volume defined by the overlap of the first and second spherical caps using a grid method 95; and 4) subtracting the volume score determined in step 3) 95 from the volume score determined in step 2) 97. The method illustrated in FIG. 8 is advantageous in that it employs both analytical and numerical methods for determining the excluded volume V_{i,j}^{E} and accordingly, is more efficient than a pure numerical approach. Still, numerical methods, such as grid methods may be employed to determine the excluded volume V_{i,j}^{E} directly.

A next step 79, determines the volume between the two spherical caps V_{1,1}, by summing the volume of rotation, V₁^{R}, determined in step 7), the volume of translation, V₁^{T}, determined in step 9), and the excluded volume V_{1,1}^{E}, determined in step 10).

A next step 81, repeats steps 4) through 11) until a volume score V_{i,j} has been determined for a plurality of spherical cap pairs that are each formed by selecting one spherical cap from the first set of spherical caps and selecting a second spherical cap whose corresponding pseudocenter is closest to the pseudocenter corresponding to the spherical cap that is selected from the first set of spherical caps. Since the volume scoring methods detailed in FIG. 5 determine the volume between two surfaces by summing the volume between spherical cap pairs, the accuracy of these methods increases as the number of spherical cap pairs that are utilized increases. Still, if less accuracy is required for a particular comparison, it is not essential that the volume between each spherical cap pair be determined. Accordingly, this step recites that the volume is determined for a plurality of spherical cap pairs

A next, and final step 83, sums the volume scores corresponding to each spherical cap pair formed in the spherical cap decomposition of the two surfaces thereby determining a volume score,

$$V = {\sum }_{i , j} {V}_{i , j}$$

(where i and j are selected subject to the constraints detailed in step 4) that reflects the volume between the two surfaces.

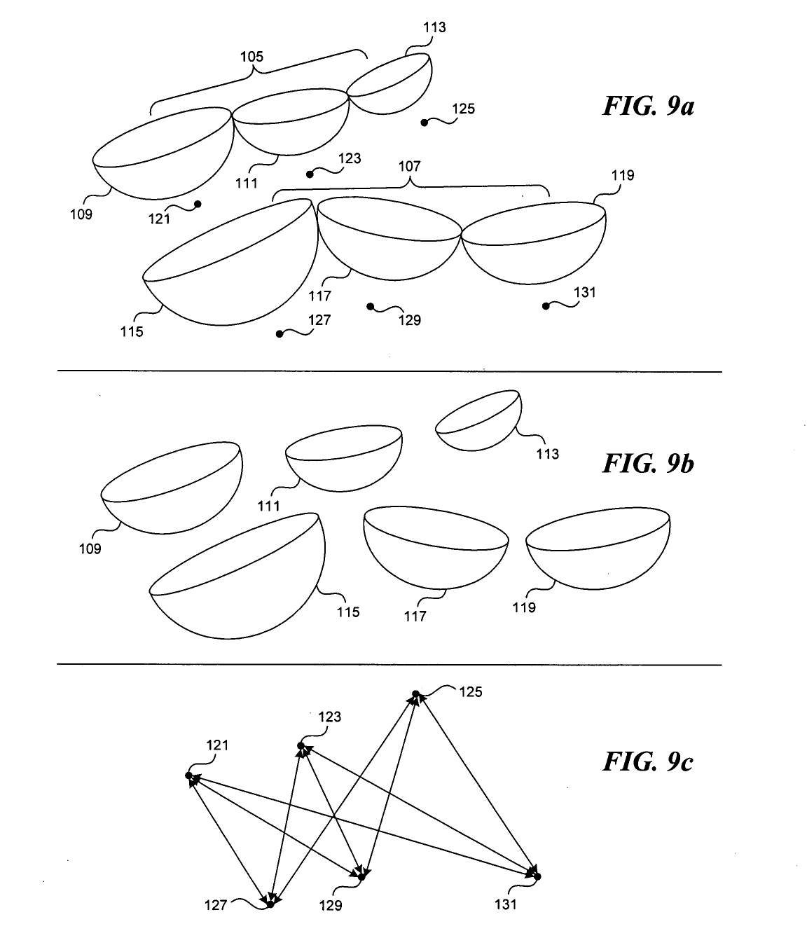

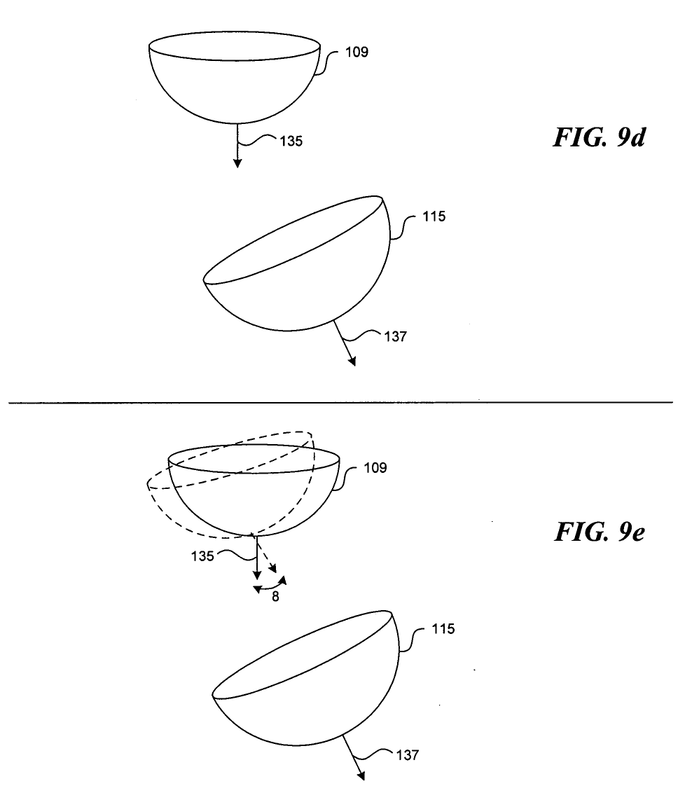

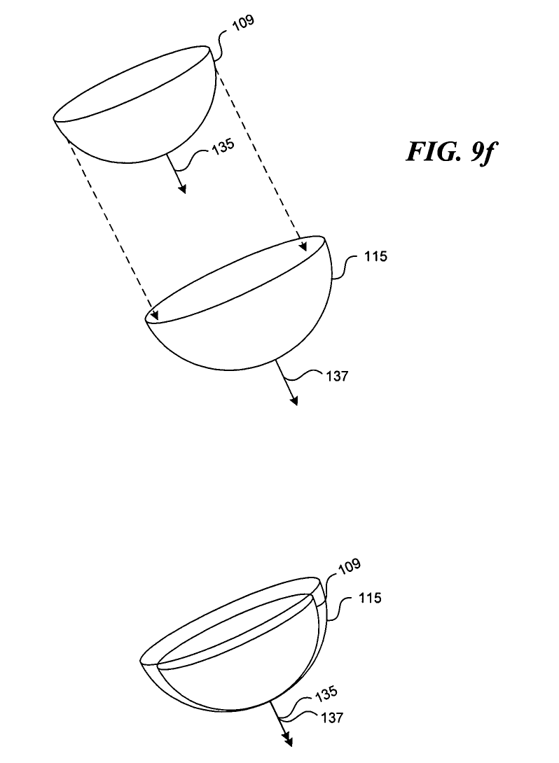

FIGS. 9 illustrates, for exemplary purposes, the application of the method illustrated in FIG. 5 for the case of a first surface and second surface each comprising three spherical caps. FIG. 9a illustrates the first surface 105, the three spherical caps 109, 111, 113, that comprise the first surface 105, their corresponding pseudocenters 121, 123, 125, the second surface 107, the three spherical caps 115, 117, 119, that comprise the second surface 107 and their corresponding pseudocenters 127, 129, 131.

A next step, illustrated in FIG. 9b decomposes both the first and second surfaces 105, 107 respectively into a first set of spherical caps Σ₁={σᵢ|i=1,2,3} 106 and a second set of spherical caps Σ₂={σⱼ|j=1,2,3}} 108.

A next step, illustrated in FIG. 9c, determines the distance between each pseudocenter 121, 123, 125 associated with the first surface 105 and each pseudocenter 127, 129, 131 associated with the second surface 107.

A next step, illustrated in FIG. 9d, selects a first spherical cap 109 from the first set of spherical caps 106 and a second spherical cap 115 from the second set of spherical caps 108 whose corresponding pseudocenter 127 is closest to the pseudocenter 121 corresponding to the spherical cap 109 selected from the first set of spherical caps 105.

A next step, also illustrated in FIG. 9d, determines a unit normal vector 135, 137 at the midpoint of each spherical cap 109, 115 selected in the prior step pointing in the direction of the solvent.

A next step, illustrated in FIG. 9e, rotates the first spherical cap 109 until its normal vector 135 is collinear with the normal vector 137 to the second spherical cap 115.

A next step, determines the volume swept by the rotation of the first spherical cap, Vᵢ₌₁^{R}=V₁^{R}.

A next step, illustrated in FIG. 9f, translates the first spherical cap 109 until its normal vector 135 is coincident with the normal vector 137 to the second spherical cap 115.

A next step, determines the volume swept by the translated first spherical cap, Vᵢ₌₁^{T}=V₁^{T}.

A next step, determines the volume of exclusion, V_{i=1,j=1}^{E}=V_{1,1}^{TE}, illustrated by the cross hatches in FIG. 9f, between the rotated, translated first spherical cap 109 and the second spherical cap 115.

A next step, sums the volume of rotation V₁^{R} of the first spherical cap, the volume of translation V₁^{T} of the first spherical cap, and the volume of exclusion V_{1,1}^{E} between the first and second spherical caps, thereby determining the volume V_{1,1} between spherical caps 109 and 115. A next step repeats steps 4)-11) in FIG. 5—i.e. determines the volumes, V_{2,2} and V_{3,3} for the two remaining pairs of spherical caps formed from 111/117 and 113/119 respectively. A final step, sums the volumes V=V_{1,1}+V_{2,2}+V_{3,3} of each of the three spherical cap pairs 109/115, 111/117, 113/119, thereby determining a volume score that represents the volume between the two surfaces 105, 107.

Methods for Determining a Chemical Similarity Score, E.

As used herein, the chemical similarity score E refers to a score that reflects the similarity in the positions and identities of the residues, or the chemical functional groups of the residues, that comprise the two functional sites. The chemical similarity score E may be based upon any monotonic function that increases (or decreases) as the number of identical residues, or chemically similar residues, that are found in the same positions in the two functional sites increases (or decreases). Chemically similar residues refer to amino acid residues that participate in similar chemical interactions. For example, all the amino acids that are hydrogen bond donors would be considered chemically similar. More generally, the chemical similarity score E may be based upon any monotonic function that increases (or decreases) as the number of identical chemical functional groups, or chemically similar functional groups, that are found in the same positions in the two functional sites increases (or decreases). Chemically similar functional groups refer to functional groups that participate in similar chemical interactions. For example, all the functional groups that are hydrogen bond donors would be considered chemically similar.

Graph methods may be used for scoring the chemical similarity of two protein surfaces. A three dimensional arrangement of physiochemical centers may be represented as a graph where the nodes of the graph correspond to physiochemical centers and the edges correspond to distances between physiochemical centers. Given two functional sites, represented by graphs A and B, comprising nodes aεA and bεB and edges d(aᵢ, aⱼ) where i, j=1, . . . |A| and d(bₖ, bₗ) where k,l=1, . . . |B|, a chemical similarity score E may be identified with the number of nodes that comprise the maximum common subgraph of A and B. Methods for determining a maximal subgraph include clique detection algorithms which are well known to those skilled in the art. Levi, G., A Note on the Derivation of Maximal Common Subgraphs of Two Directed or Undirected Graphs, Calcolo 9, 341-354 (1973). One method for determining a chemical similarity score based on a clique detection algorithm comprises the steps of: 1) determining a plurality of node pairs (aᵢ, bₖ) such that each node in the pair corresponds to the same type of physiochemical center as the other—e.g. hydrogen bond donor; 2) determining a set of nodes c_{i,k} such that each node corresponds to a node pair (aᵢ, bₖ); 3) determining an edge between any two nodes c_{i,k} and c_{j,l} if d(aᵢ,aⱼ)≈d(bₖ,bₗ), thereby determining a graph C; 4) determining the maximum common subgraph of C using a clique detection algorithm, such as the Bron-Kerbosch algorithm; and 5) identifying the chemical similarity score with the number of nodes that comprise the maximal common subgraph of C. Bron, G., Kerbosch, J. Finding All Cliques in an Undirected Graph, Comm. ACM, 16: 5747-577 (1982).

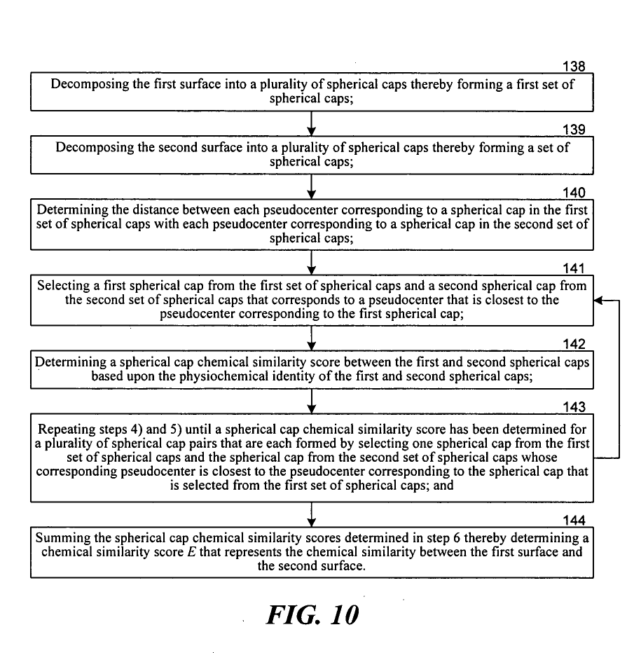

The methods illustrated in FIG. 2 represents each or substantially each physiochemical center on the surface of a protein with one or more corresponding spherical caps (via their corresponding pseudocenters). FIG. 10 illustrates another method according to the invention for scoring the chemical similarity of two proteins represented by the methods illustrated in FIG. 2 comprising the steps of: 1) decomposing the first surface into a plurality of spherical caps thereby forming a first set of spherical caps 138; 2) decomposing the second surface into a plurality of spherical caps thereby forming a set of spherical caps 139; 3) determining the distance between each pseudocenter corresponding to a spherical cap in the first set of spherical caps and each pseudocenter corresponding to a spherical cap in the second set of spherical caps 140; 4) selecting a first spherical cap from the first set of spherical caps and a second spherical cap from the second set of spherical caps whose corresponding pseudocenter is closest to the pseudocenter corresponding to the first spherical cap 141; 5) determining a spherical cap chemical similarity score between the first and second spherical caps based upon the physiochemical identity of the first and second spherical caps 142 as determined from the physiochemical identity of their corresponding pseudocenters; 6) repeating steps 4) 141 and 5) 142 until a spherical cap chemical similarity score has been determined for a plurality of spherical cap pairs that are each formed by selecting one spherical cap from the first set of spherical caps and the spherical cap from the second set of spherical caps whose corresponding pseudocenter is closest to the pseudocenter corresponding to the spherical cap that is selected from the first set of spherical caps 143; and 7) summing the spherical cap chemical similarity scores determined in step 6) thereby determining a chemical similarity score that represents the chemical similarity between the first surface and the second surface 144.

The first four steps, 138-141, in the methods illustrated in FIG. 10 correspond to the first four steps of the methods illustrated in FIG. 5 and detailed above.

The fifth step, 142, determines a spherical cap chemical similarity score between the first and second spherical caps based upon their physiochemical identity. As used herein, a spherical cap chemical similarity score refers to a score that represents the physiochemical similarity of two spherical caps. One suitable method for determining a spherical cap chemical similarity scores uses binary scoring. If the first and second spherical caps are characterized by the same or physiochemically compatible properties as defined by their corresponding pseudocenters, the pair is scored +1; otherwise, the pair is score 0. In addition to binary scoring schemes, a spherical cap chemical similarity score may use scoring schemes that reflect the relative physiochemical similarity of two spherical caps along a continuum. One such scheme illustrated in Table 2, scores the physiochemical similarity between any two naturally occurring amino acid residues from 1.1 to 6, with 6 indicating identity and 1.1 indicating the most physiochemically non-compatible residue pair. Another scheme for scoring the chemical similarity of a pair of spherical caps based upon their corresponding pseudocenters is provided in Table 3.

TABLE 3
provides a scoring matrix for scoring the chemical similarity of a
spherical cap pair based upon the physiochemical identities of the
corresponding pseudocenters.

| Spherical Cap | | | Mixed | Hydro- | Hydro- |
|---|---|---|---|---|---|
| Pseudocenter | H-Bond | H-Bond | Donor | phobic | phobic |
| Type | Donor | Acceptor | Acceptor | Aliphatic | Aromatic |
| H-Bond | 1 | 0 | 1 | 0 | 0 |
| Donor | | | | | |
| H-Bond | 0 | 1 | 1 | 0 | 0 |
| Acceptor | | | | | |
| Mixed Donor | 1 | 1 | 1 | 0 | 0 |
| Acceptor | | | | | |
| Hydrophobic | 0 | 0 | 0 | 1 | 1 |
| Aliphatic | | | | | |
| Hydrophobic | 0 | 0 | 0 | 1 | 1 |
| Aromatic | | | | | |

A next step, 143, repeats the fourth 141 and fifth steps 142, until a spherical chemical similarity score is determined for a plurality of spherical cap pairs that are each formed by selecting one spherical cap from the first set of spherical caps and selecting a second spherical cap whose corresponding pseudocenter is closest to the pseudocenter corresponding to the spherical cap that is selected from the first set of spherical caps. Since the chemical similarity scoring methods detailed in FIG. 10 determine the chemical similarity between two protein surfaces by determining the chemical similarity pairwise between the spherical cap elements that comprise the respective surfaces, the accuracy of these methods increases as the percentage of the two surfaces is sampled. Still, if less accuracy is required for a particular comparison, it is not essential that the chemical similarity between each spherical cap pair be determined. Accordingly, this step recites that a spherical cap chemical similarity score is determined for a plurality of spherical cap pairs. Since, the chemical similarity scoring methods detailed in FIG. 10 determine the chemical similarity of two surfaces by summing the chemical similarity between spherical cap pairs, the accuracy of these methods increases as the number of spherical cap pairs that are utilized increases. Still, if less accuracy is required for a particular comparison, it is not essential that the chemical similarity between each spherical cap pair be determined. Accordingly, this step recites that the chemical similarity are determined for a plurality of spherical cap pairs

A final step 144, sums the spherical cap chemical similarity scores determined in step 6) 143 thereby determining a chemical similarity score, E, of the two protein surfaces.

Exemplary Types of Physiochemical Similarity Scores that may be Formed from E and V.

A general functional form for relating S to V and E is S=ƒ(VE) where ƒ(VE) is a function that depends upon V and E. One suitable form of the physiochemical score S is S=Eƒ(V) where

$$f ( V ) = \frac{1}{β ( 1 + {ⅇ}^{γ V} )} .$$

Another suitable form for S, is S=Vƒ(E) where

$$f ( E ) = \frac{1}{β ( 1 + {ⅇ}^{γ E} )} .$$

V may be determined by any method for determining the volume between two surfaces including numerical methods or, if the surfaces are represented according to the methods illustrated in FIG. 2, by using the volume scoring methods illustrated in FIG. 5. Similarly, E may be determined by any method that scores the similarity in the positions and identities of the residues that comprise two proteins. Accordingly, graph methods may be applied to determine E or if the two surfaces are represented according to the methods illustrated in FIG. 2, the chemical similarity scoring methods illustrated in FIG. 10 may also be applied. γ controls the rate of falloff of the scaling function ƒ(V) and β controls the y-offset.

For the methods, such as those illustrated in FIG. 2, that represent the two surfaces, A and B, as a plurality of finite surface area elements aε{1 . . . a′} and bε{1 . . . b′} where the allowed simultaneous values of a and b are limited by a set of constraints (e.g. step 3 in the methods of FIG. 5), another form of S is

$$S = {\sum }_{{a = 1}_{b = 1} ,}^{{a}^{′} , {b}^{′}} {S}_{a , b}$$

where S_{a,b}=E_{a,b}ƒ(V_{a,b}) for all allowed simultaneous values of a and b. A further refinement on this scoring scheme provides a scoring penalty gap, if the distance between two physiochemical centers d(a,b), exceeds a threshold distance dₜₕ. One way of implementing such a penalty is S_{a,b}=E_{a,b}ƒ(V_{a,b}) if d(a,b)≦dₜₕ, otherwise, S_{a,b}=gap(1−ƒ(V_{a,b}))where gap<0. gap preferably ranges from 0 to −1, with −0.2 to −0.8 more preferred, and with −0.04 to −0.06 still more preferred. β preferably ranges from 0.5 to 2, with 0.6 to 1.5 more preferred and with 0.9 to 1.1 still more preferred. γ preferably ranges from 1.0 to 3 with 1.5-2.5 more preferred and 2.1 to 2.3 still more preferred.

Since the methods illustrated in FIGS. 5 and 10 sum the individual volume and chemical similarity scores for each spherical cap pair, these methods may easily be applied to determine a physiochemical similarity score from

$$S = {\sum }_{{a = 1}_{b = 1} ,}^{{a}^{′} , {b}^{′}} {S}_{a , b} .$$

The following example will illustrate how to calculate a physiochemical score, S, from

$$S = {\sum }_{{a = 1}_{b = 1} ,}^{{a}^{′} , {b}^{′}} {S}_{a , b}$$

where S_{a,b}=E_{a,b}ƒ(V_{a,b}) if d(a,b)≦dₜₕ, otherwise, S_{a,b}=gap(1−ƒ(V_{a,b})) and using the example illustrated in FIG. 9.

FIG. 9a illustrates a first surface 105, the three spherical caps 109, 111, 113, that comprise the first surface 105, their corresponding pseudocenters 121, 123, 125, a second surface 107, the three spherical caps 115, 117, 119, that comprise the second surface 107 and their corresponding pseudocenters 127, 129, 131. Assume that the pseudocenters 121, 123, 125 and their corresponding spherical caps 109, 111, 113 are respectively a hydrogen bond donor, a hydrogen bond acceptor and a mixed donor receptor. Further assume that the pseudocenters 127, 129, 131 and their corresponding spherical caps 115, 117, 119 are respectively a hydrogen bond donor, a hydrogen bond acceptor and a hydrophobic center. For this example, a binary chemical similarity scoring scheme is used that scores two chemically identical or chemically compatible spherical caps, with the score of “1” and scores two distinct or chemically non-compatible spherical caps with the score of “0”. Compatible spherical caps refer to distinct but energetically compatible residues/functional groups such as a hydrogen bond donor and a mixed donor acceptor. Chemically non-compatible physiochemical centers refer to distinct and energetically non-compatible residues/functional groups such as a hydrogen bond acceptor and a hydrophobic center.

A first step, illustrated in FIG. 9b decomposes both the first and second surfaces 105, 107 respectively into a first set of spherical caps Σ₁={σᵢ|i=1, 2, 3} 106 and a second set of spherical caps Σ₂={σⱼ|j=1, 2, 3} 108.

A second step, illustrated in FIG. 9c, determines the distance between each pseudocenter 121, 123, 125 associated with the first surface 105 and each pseudocenter 127, 129, 131 associated with the second surface 107.

A third step, illustrated in FIG. 9d, selects a first spherical cap 109 from the first set of spherical caps 106 and a second spherical cap 115 from the second set of spherical caps 108 whose corresponding pseudocenter 127 is closest to the pseudocenter 121 corresponding to the spherical cap 109 selected from the first set of spherical caps 105.

A fourth step, also illustrated in FIG. 9d, determines a unit normal vector 135, 137 at the midpoint of each spherical cap 109, 115 selected in the prior step pointing in the direction of the solvent.

A fifth step, illustrated in FIG. 9e, rotates the first spherical cap 109 until its normal vector 135 is collinear with the normal vector 137 to the second spherical cap 115.

A sixth step, determines the volume swept by the rotation of the first spherical cap.

A seventh step, illustrated in FIG. 9f, translates the first spherical cap 109 until its normal vector 135 is coincident with the normal vector 137 to the second spherical cap 115.

An eighth step, determines the original distance between the first and second spherical caps before the rotation and translation of the first spherical cap based upon the degree of rotation and translation of the first spherical cap. This step will be used in the subsequent steps to determine the spherical cap chemical similarity score E_{1,1}.

A ninth step, determines the volume swept by the translated first spherical cap, V₁^{T}.

A tenth step, determines the volume of exclusion, V_{1,1}^{E}, between the rotated, translated first spherical cap 109 and the second spherical cap 115.

An eleventh step, sums the volumes determined corresponding to the rotation V₁^{R} and translation V₁^{T} of the first spherical cap and the volume of exclusion V_{1,1}^{E}, illustrated in FIG. 9f thereby determining the volume V_{1,1} between spherical caps 109 and 115.

A twelfth step, determines the spherical cap chemical similarity score E_{1,1} between the first and second spherical caps based upon their physiochemical identity and the original distance between the two spherical caps. If the original distance between the first and second spherical caps is less than the threshold distance, dₜₕ, E_{1,1} may be assigned a score of 1 since the first and second spherical caps are both hydrogen bond donors. Under this assumption, the physiochemical similarity score for the first and second spherical caps is S_{1,1}=1·ƒ(V_{1,1}) where ƒ(V_{1,1})=1/β(1+exp(γV_{1,1})). If the original distance between the first and second spherical caps is greater than the threshold distance, dₜₕ, then S_{1,1}=gap(1−ƒ(V_{1,1})) where gap<0. While this example determines the initial distance between the first and second spherical caps based upon the degree of rotation and translation of the first spherical cap, other schemes for determining this initial distance may used. Another suitable scheme determines the midpoints of the first and second spherical caps and identifies the distance between the midpoints as the initial distance between the first and second spherical caps. As used herein, the midpoint of a spherical cap refers to the point at R−h/2 in FIG. 5. Alternatively, the original distance between the first and second spherical caps may be determined from the distance between their corresponding pseudocenters. In addition to using a distance threshold, a volume score threshold may also be used since the volume score between two spherical caps increases proportionately as the distance between two spherical caps increases.

A next step repeats steps 3)-12)—i.e. determines the physiochemical similarity scores, S_{2,2} and S_{3,3} for the two remaining pairs of spherical caps formed from 111/117 and 113/119 respectively. A final step, sums the physiochemical similarity scores between each of three spherical cap pairs, S=S_{1,1}+S_{2,2}+S_{3,3} formed from 109/115 , 111/117 and 113/119, thereby determining a physiochemical similarity score, S, that represents the physiochemical similarity between the two surfaces 105, 107. Assuming the distance between each spherical cap pair is less than dₜₕ, S=1·ƒ(V_{1,1})+·1·ƒ(V_{2,2})+0. The second and third terms follow from the fact that the second spherical cap pair, 111/117, comprises spherical caps of the same physiochemical type, while the third pair, 113/119 comprises spherical caps of different physiochemical types.

Although this example determined the volume score between two spherical caps, V_{i,j}, before determining the corresponding chemical similarity score E_{i,i} it will be appreciated from S_{i,j}=E_{i,j}ƒ(V_{i,j}), that E_{i,j}, may be determined first. It will further be appreciated that while this example used a binary chemical similarity scoring scheme and only 4 types of physiochemical centers (hydrogen bond donor, hydrogen bond acceptor, mixed donor receptor and hydrophobic center) other chemical similarity scoring schemes, such as the one illustrated in Table 2, may also be used.

Methods for Varying the Relative Orientation of Two Surfaces-11

One embodiment of the invention represents both surfaces in an arbitrary coordinate system and rigidly transforms one or both surfaces relative to the other in order to sample the relative orientation space between the two surfaces. Either the underlying structural coordinates that are used as a basis for determining the mathematical surfaces that represent two functional sites may be rigidly transformed or the surfaces may be rigidly transformed. As used herein a rigid body transformation refers to a rotation or translation of a discrete or continuous function as a whole. As used herein the relative orientation of two surfaces refers to the mathematical relationship of one surface relative to the other in a common reference frame. As used herein the relative orientation space refers to the set of all allowed relative orientations that may be formed between the two surfaces. This brute force method iteratively rotates and translated each surface relative to the other without regard to the degree of overlap between the surfaces. Although straightforward to implement, this method is computationally inefficient since it may be expected that for many of the relative orientations there would be little to no overlap between the surfaces and the physiochemical similarity scores for such relative orientations would be negligible.

Surfaces may be rigidly rotated/translated relative to each other using deterministic or random methods. There is no inherent limitation on how the relative orientation of the two surfaces may be varied. Random methods such as Monte Carlo methods, simulated annealing methods, genetic algorithms, reinforced learning methods, or recursive linear estimate methods may be used to rotate/translate the surfaces. The following mathematically equivalent transformations may be used for varying the orientation of the first surface relative to the second surface: 1) the first surface may be rotated/translated while fixing the second surface; 2) both surfaces may be translated/rotated; 3) the coordinate system used to represent one surface may be varied while fixing the coordinate system used to represent the other surface or 4) both coordinate systems used to represent the two surfaces may be varied.

A rigid transformation in R³ is an orientation-preserving isometry in three dimensional Euclidean space. Every rigid transformation of a surface may be decomposed in to a rotation and translation. In general, any rotation can be decomposed into three sequential Euler rotations. In R³, the Euler rotation matrices are given by:

$$\begin{matrix}{R}_{x} ( α ) = [ \begin{matrix}1 & 0 & 0 \\ 0 & cos α & sin α \\ 0 & - sin α & cos α\end{matrix} ] & Eq . 1 \\ {R}_{y} ( β ) = [ \begin{matrix}cos β & 0 & - sin β \\ 0 & 1 & 0 \\ sin β & 0 & cos β\end{matrix} ] & \\ {R}_{z} ( γ ) = [ \begin{matrix}cos γ & sin γ & 0 \\ - sin γ & cos γ & 0 \\ 0 & 0 & 1\end{matrix} ] & \end{matrix}$$

where x, y, z represent the axes of rotation and α, β, γ, represent the angles of the rotation. It may be shown, the rigid rotation R and translation T of a point x on a surface to x′ may be expressed as

$$\begin{matrix}{x}^{′} = R x + T & Eq . 2 \\ [ \begin{matrix}{x}_{1}^{′} \\ {x}_{2}^{′} \\ {x}_{3}^{′}\end{matrix} ] = [ \begin{matrix}{r}_{1 , 1} & {r}_{1 , 2} & {r}_{1 , 3} \\ {r}_{2 , 1} & {r}_{2 , 2} & {r}_{2 , 3} \\ {r}_{3 , 1} & {r}_{3 , 2} & {r}_{3 , 3}\end{matrix} ] [ \begin{matrix}{x}_{1} \\ {x}_{2} \\ {x}_{3}\end{matrix} ] + [ \begin{matrix}{t}_{1} \\ {t}_{2} \\ {t}_{3}\end{matrix} ] & Eq . 3\end{matrix}$$

where R is an orthonormal 3-by-3 rotation matrix with unit determinant and T is a 3 vector. If a surface Σ comprising n residues, each with coordinate xᵢ, is represented as Σ={σᵢ(xᵢ)|i=1 . . . n} the transformation of Σ by an arbitrary rotation and translation may be found may be found from Σ={σᵢ(x′ᵢ)|i=1 . . . n} where x′ᵢ=Rxᵢ+T. Thus, the full relative orientation phase space between two surfaces may be sampled by incrementally translating and rotating each surface using the above equations. While this method may be implemented straightforwardly, it is computationally inefficient since it entails rotating/translating the rigid surface in three dimensions. More importantly, for many of the relative orientations that may be formed between two surfaces, it would be expected that there would be no overlap between the surfaces. Accordingly, more efficient methods should sample the relative orientation phase space between the two surfaces so that only those relative orientations that produce some overlap are considered.

Determining a Plurality of Physiochemical Similarity Scores Corresponding to a Plurality of Relative Orientations of the First and Second Surfaces-13, 15

Having determined a second orientation between the two surfaces, a next step in the methods illustrated in FIG. 1 13, determines the corresponding physiochemical similarity score between the two surfaces. This process of selecting a new orientation between the two surfaces and determining the corresponding physiochemical similarity score S between the surfaces is repeated a plurality of times 15. There is no inherent limitation on the number of physiochemical similarity score determinations that must be made by the surface comparison methods according to the invention. As one ordinarily skilled in the art would appreciate, the sensitivity of the methods according to the invention increases as the number of physiochemical similarity scores increases, and accordingly, the number of orientations between the two surfaces increases.

Methods for Determining a Protein Similarity Score from a Plurality of Physiochemical Similarity Scores-19, 21

To determine a protein similarity score between two surfaces, the physiochemical similarity scores corresponding to multiple orientations 15 of the two surfaces may be ranked 19 from most similar to least similar. For the case where S increases with increasing similarity of the two functional sites, the protein similarity score for a given pair of functional sites may be identified with the maximum value of S. While the maximum value of S is the preferred metric for comparing two functional sites in this case, sub-maximal values of S may still be used, albeit with decreased accuracy.

Alternatively, for those methods where the volume score, V, and the chemical similarity score, E, decrease with increasing similarity of the two proteins, the protein similarity score for a given pair of functional sites may be identified with the minimum value of S. Once again, for the reasons outlined immediately above, a super-minimum protein similarity score still provides useful information.

Graph Based Methods for Determining the Similarity Between Two Functional Sites.

One embodiment of the invention iteratively varies the relative orientation between the two surfaces, via rigid body transformations of one or both surfaces, without regard to whether a given relative orientation produces any overlap between the two surfaces. While this “brute force” method is straightforward to implement it is computationally inefficient since may potential relative orientations between two surfaces correspond to no overlap, and thus, a zero similarity score.

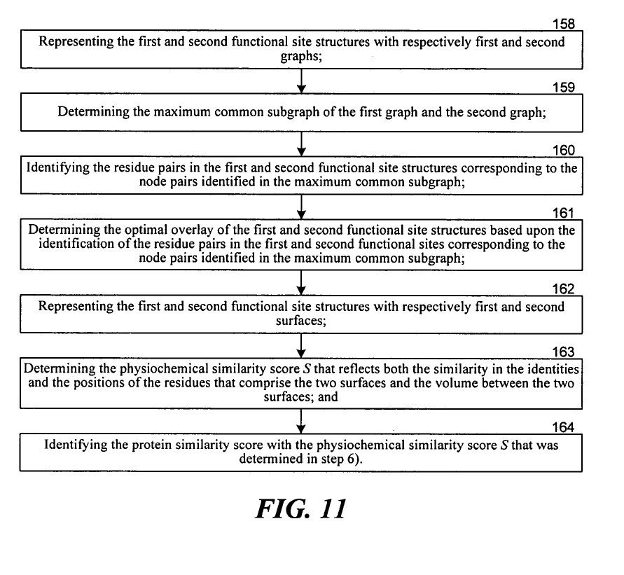

Variations of the method of FIG. 1 may use graph based methods to efficiently identify those relative orientations between the two surfaces that are likely to correspond to significant physiochemical similarity scores before computing the physiochemical similarity scores according the invention. FIG. 11 illustrates one such variation of the method of FIG. 1 comprising the steps of: 1) representing the first and second functional sites with respectively first and second graphs 158; 2) determining the maximum common subgraph of the first graph and the second graph 159; 3) identifying the residue pairs in the first and second functional sites corresponding to the node pairs identified in the maximum common subgraph 160; 4) determining the optimal overlay of the first and second functional sites based upon the identification of the residue pairs in the first and second functional sites corresponding to the node pairs identified in the maximum common subgraph 161; 5) representing the optimally overlayed first and second functional sites with respectively first and second surfaces 162; 6) determining the physiochemical similarity score S that reflects both the similarity in the identities and positions of the residues that comprise the two sites and the volume between the first and the second surfaces 163; and 7) identifying a protein similarity score with the physiochemical similarity score S that was determined in step 6) 164. As one skilled in the art will recognize that by calculating the physiochemical similarity score for only one relative conformation between the two surfaces, substantial computational efficiencies may be gained.

The first step 158 may sufficiently employ the methods for representing a functional site with a graph detailed in the subsection entitled Graph Based Methods within the section entitled Methods for Determining a Surface that Represents the Physiochemical Topography of a Functional Site.

The second step 159 may sufficiently determine the maximum common subgraph or clique using any of the method know in the art for determining the maximum common subgraph or clique of two graphs such as the Bron-Kerbosch algorithm. C. Bron, J. Kerbosch. Algorithm 457: Finding all cliques of an undirected graph, Communications of the Association for Computing Machinery, 16(9):575-577, (1973). See also Carraghan, R., Pardalos, P. M. An Exact Algorithm for the Maximum Clique Problem, 9, 375-382 (1990). One embodiment of the invention uses the algorithm detailed in Carraghan, R., Pardalos, P. M. An Exact Algorithm for the Maximum Clique Problem, 9, 375-382 (1990) for determining the maximum common clique between the two graphs that represent the two functional sites.

Given two protein surfaces, represented by graphs A and B, comprising nodes aεA and bεB, and edges d(aᵢ,aⱼ) where i,jε{1, . . . |A|} and d(bₖ,bₗ)where k,lε{1, . . . |B|}, a common subgraph between A and B is determined by a method comprising the steps of: 1) determining a plurality of node pairs (aᵢ, bₖ) such that each node in the pair corresponds to the same residue type (or sidechain functional group type); 2) determining a third graph C comprising set of nodes c_{i,k}, wherein each node c_{i,k} corresponds to a node pair (aᵢ, bₖ), and a set of edges between any two nodes c_{i,k} and c_{j,l} if d(aᵢ, aⱼ)=d(bₖ,bₗ); and 3) identifying a set of nodes connected by edges in C as a common subgraph of A and B. The maximum common subgraph of A and B is the subgraph of C with maximum number of nodes. The output of a common subgraph algorithm or common clique comprises a set of node pairs—i.e. two sets of points connected by a bijection. Each node pair comprises one node from one surface and its corresponding “overlaying” node in the second surface.

The residues in each functional site that correspond to the nodes of the maximum common subgraph may be identified based upon the structures of the two functional sites, the graphs of the two functional sites and the maximum common subgraph 160. For example, for the case where graph nodes correspond to residues in the functional sites, the residues in each functional site that correspond to the nodes of the maximum common subgraph may be identified by mapping each residue aᵢ and bₖ, of each maximum common subgraph node c_{i,k}, to their corresponding functional sites. If instead, graph nodes represent chemical functional groups, the chemical functional groups in each functional site that correspond to the nodes of the maximum common subgraph may be identified by mapping each chemical functional group aᵢ and bₖ, of each maximum common subgraph node c_{i,k}, to their corresponding functional sites. The corresponding residues of each functional site may be identified by reference to the functional groups mapped to each functional site, the identities of the residues that comprise each functional site, the structure of each residue, and the structure of each functional site.

As used herein, the optimal overlay between the first and second functional sites refers to the relative orientation between two functional site structures that minimizes the rms distance between overlaying residues when the two functional site structures are overlayed. Assume that two overlayed functional sites, U and W, are represented as U={uᵢ|i=1 . . . n} and W={wᵢ|1 . . . n} where uᵢ and wᵢ represent the positions of overlaying residues on the respective functional sites. uᵢ and wᵢ may be identified respectively aᵢ and bₖ, of each node pair in the maximum common subgraph.

One measure of the similarity of the two functional sites U and W is the root mean square distance between the residues (or sidechain functional groups)

$$d ( U , W ) = {( \frac{1}{n} {\sum }_{i} {\vert {u}_{i} - {w}_{i} \vert}^{2} )}^{1 / 2} \qquad Eq . 4$$

Thus, the problem of determining the optimal overlay between two functional sites U and W, or the surfaces that represent them reduces to determining the optimal rigid transformation of U that minimizes the RMS distance between U and W 161. As used herein the optimal rigid transformation refers the transformation of U that minimizes the RMS distance between U and W. As one skilled in the art will appreciate, the choice of transforming U is arbitrary, W could also be transformed to minimize the RMS distance between U and W. Any rigid transformation can be decomposed into a translation followed by, or preceded by, a rotation. Thus, the problem of determining the optimal rigid transformation of U is equivalent to determining the optimal rigid translation, tₒₚₜ, of U and the optimal rigid rotation, qₒₚₜ, of U.

The optimal translation, tₒₚₜ, of U translates \overline{U}, the centroid of U, to \overline{W}, the centroid of W, where

$$\overline{U} = \frac{1}{n} {\sum }_{i} {u}_{i} and \overline{W} = \frac{1}{n} {\sum }_{i} {w}_{i} .$$

For simplicity of presentation, assume that \overline{U}=\overline{W}=0. Since the optimal rotation qₒₚₜ, rotates U such the RMS distance between U and W is minimized, determining the optimal rotation requires minimizing

$$\begin{matrix}F ( q ) = {\sum }_{i} {\vert q {u}_{i} {q}^{*} - {w}_{i} \vert}^{2} & Eq . 5 \\ = {\sum }_{i} {\vert {u}_{i} \vert}^{2} - 2 〈 q {u}_{i} {q}^{*} , {w}_{i} 〉 + {\vert {w}_{i} \vert}^{2} & \end{matrix}$$

where q is a unit quaternion, uᵢ is an imaginary quaternion and wᵢ is an imaginary quaternion. It will be appreciated by one ordinarily skilled in the art that quᵢq* represents the rotation of the residue located at position uᵢ. |uᵢ|² and |wᵢ|² are scalars and so are unaffected by rotation. Thus, minimizing Equation 5 is equivalent to maximizing

$${\sum }_{i} 〈 q {u}_{i} {q}^{*} , {w}_{i} 〉 . \qquad Eq . 6$$

Since it may be shown that multiplication with a unit quaternion preserves scalar products, <quᵢq*,wᵢ>=<quᵢ,q*wᵢ>. It may also be shown that

$$\begin{matrix}q {u}_{i} = [ \begin{matrix}0 & - {u}_{i , 1} & - {u}_{i , 2} & - {u}_{i , 3} \\ {u}_{i , 1} & 0 & {u}_{i , 3} & - {u}_{i , 2} \\ {u}_{i , 2} & - {u}_{i , 3} & 0 & {u}_{i , 1} \\ {u}_{i , 3} & {u}_{i , 2} & - {u}_{i , 1} & 0\end{matrix} ] q = {U}_{i} q & Eq . 7 \\ {w}_{i} q = [ \begin{matrix}0 & - {w}_{i , 1} & - {w}_{i , 2} & - {w}_{i , 3} \\ {w}_{i , 1} & 0 & {w}_{i , 3} & - {w}_{i , 2} \\ {w}_{i , 2} & {w}_{i , 3} & 0 & - {w}_{i , 1} \\ {w}_{i , 3} & - {w}_{i , 2} & {w}_{i , 1} & 0\end{matrix} ] q = {W}_{i} q & Eq . 8\end{matrix}$$

Equation 5 may be rewritten as

$${\sum }_{i} 〈 {U}_{i} q , {W}_{i} q 〉 = {\sum }_{i} {q}^{T} {U}_{i}^{T} {W}_{i} q = {q}^{T} ( {\sum }_{i} {U}_{i}^{T} {W}_{i}^{T} ) q = {q}^{T} C q \qquad Eq . 9$$

C is a square, symmetric matrix. Accordingly, C has four real eigenvalues, λ₁≧λ₂≧λ₃≧λ₄. The corresponding eigenvectors are pairwise orthogonal and span R⁴. Thus any quaternion q may be written as

$$\begin{matrix}q = {\sum }_{j = 1}^{4} {x}_{j} {e}_{j} . \\ Hence , & Eq . 10 \\ {q}^{T} C q = 〈 q , {\sum }_{j = 1}^{4} {x}_{j} {λ}_{j} {e}_{j} 〉 = {\sum }_{j = 1}^{4} {x}_{j}^{2} {λ}_{j} . & Eq . 11\end{matrix}$$

If only unit quaternions are of interest,

$${\sum }_{j = 1}^{4} {x}_{j} = 1 ,$$

and q^{T}Cq≦λ₁. The maximum of this inequality, q^{T}Cq=λ₁, uniquely occurs for xₗ. Thus, the optimal rotation that minimizes Equation 5 is identified with qₒₚₜ=q=e₁. In other words the optimal rotation of U is identified with the unit vector eⱼ that corresponds to the largest eigenvalue λⱼ.

In general, a vector in R³, represented as quaternion r may be transformed according to
r′=qrq*+t Eq. 12
where q is a quaternion, q* is the complex conjugate of q, and t is a quaternion that translates the rotated vector r. Thus, the optimal overlay of U and W may be determined by optimally transforming each coordinate vector uᵢ according to:
u_{i,opt}=qₒₚₜuᵢq*ₒₚₜ+tₒₚₜ Eq. 13

The fifth step 162, may sufficiently employ the methods according to the invention for determining a surface that represents the physiochemical topography of a functional site detailed in the section entitled Methods for Determining a Surface that Represents the Physiochemical Topography of a Functional Site-3, 5. In one embodiment of the invention according to FIG. 11, the first and second surfaces are represented using the method illustrated in FIG. 2.

The sixth step 163, of the method illustrated in FIG. 11 may sufficiently use the methods for determining a physiochemical similarity score S detailed in the section entitled Methods for Determining a Physiochemical Similarity Score, S-9. While the surface comparison method according to the invention in FIG. 11 uses a physiochemical similarity score, S, the method may alternatively also score the similarity of two functional sites using the chemical similarity score, E.

Further variations on the method illustrated in FIG. 11 may use sub-maximum common subgraphs instead of the maximum common subgraph. For example, if one or more sub-maximum common subgraphs are determined, physiochemical similarity scores may be calculated for each relative orientation between the two surfaces corresponding to a sub-maximum common subgraph. The physiochemical similarity scores are then ordered from most similar to least similar and the physiochemical similarity score corresponding to the highest degree of similarity is then identified as the similarity score for the two surfaces.

Geometric Hashing Based Methods for Determining the Similarity Between Two Protein Surfaces

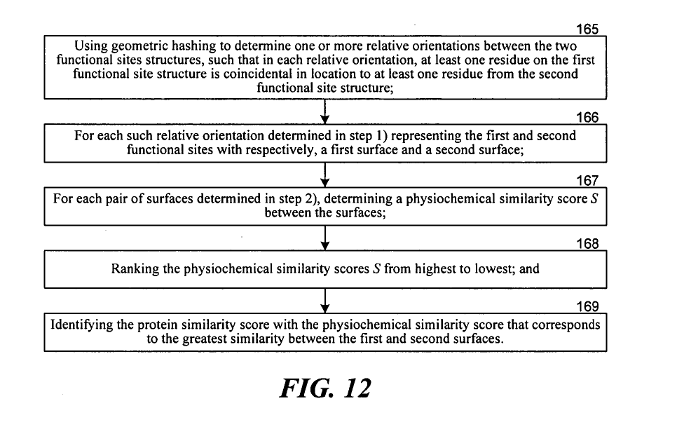

Variations of the method of FIG. 1 may use geometric hashing based methods to efficiently identify the relative orientations between the two surfaces that are likely to correspond to significant physiochemical similarity scores before computing the physiochemical similarity scores according the invention. FIG. 12 illustrates one such variation of the method of FIG. 1 comprising the steps of: 1) using geometric hashing to determine one or more relative orientations between the two functional site structures, such that in each such relative orientation, at least one residue from the first functional site is coincidental in location to at least one residue from the second functional site 165; 2) for each such relative orientation determined in step 1) representing the first and second functional sites with respectively, a first surface and a second surface 166; 3) for each pair of surfaces determined in step 2), determining a physiochemical similarity score S between the surfaces 167; 4) ranking the physiochemical similarity scores from highest to lowest 168; and 5) identifying the protein similarity score with the physiochemical similarity score that corresponds to the greatest similarity between the first and second surfaces 169.

The first step 165, in the method illustrated in FIG. 12 uses geometric hashing to efficiently determine a plurality of relative orientations between two surfaces, such that in each relative orientation at least one residue from the first functional site is coincidental in location to at least one residue from the second functional site. In other words, geometric hashing is used to efficiently determine the relative orientations between the two surfaces corresponding to at least some overlap of the two corresponding surfaces. One geometric hashing based method for determining one or more relative orientations such that in each relative orientation, at least one residue on the first functional site is coincidental in location to at least one residue from the second functional site comprises the steps: 1) determining a first hash table for the first functional site structure, wherein each hash table entry comprises the identification of a coordinate system and the coordinates of one residue of the first site; 2) determining a second hash table for the second site; wherein each hash table entry comprises the identification of a coordinate system and the coordinates of one residue of the second site 3) selecting a first entry from the first hash table and second entry from the second hash table; 4) comparing the first entry from the first has table to each, or substantially each other entry in the second hash table; and 5) for each pair of entries where the two coordinate systems are identical and where the coordinates of the single residue are identical, identifying said coordinate system as a coordinate system where the relative orientation between the two sites produces some overlap.

Once a coordinate system has been identified that corresponds to a relative orientation that produces some overlap between the two sites, the first and second functional sites are transformed into that coordinate system. If a protein surface Σ comprising n residues σ, each with coordinate xᵢ, is represented as Σ={σᵢ(xᵢ)|1 . . . n}, the transformation of Σ into a new coordinate system (e₁, e₂, e₃) may be represented as Σ′={σᵢ(x′ᵢ)|1 . . . n}, where x′ᵢ=(xᵢ·e₁,xᵢ·e₂,xᵢ·e₃).

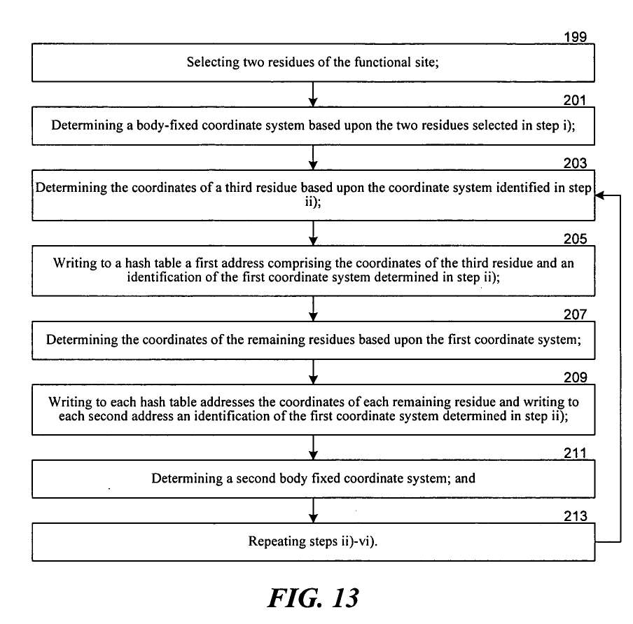

Methods for building hashing tables are well known in the art. One method, illustrated in FIG. 13, for building a hash table that represents a functional site comprising n residues comprises the steps of: i) selecting two residues of the functional site 199; ii) determining a body-fixed coordinate system based upon the two residues selected in step i) 201; iii) determining the coordinates of a third residue based upon the coordinate system identified in step ii) 203; iv) writing to a hash table a first address comprising the coordinates of the third residue and an identification of the first coordinate system determined in step ii) 205; v) determining the coordinates of the remaining residues based upon the first coordinate system 207; vi) writing to each first table address the coordinates of each remaining residue and writing to each second address an identification of the first coordinate system determined in step ii) 209; vii) determining a second body fixed coordinate system 211; and viii) repeating step ii)-vi) 213.

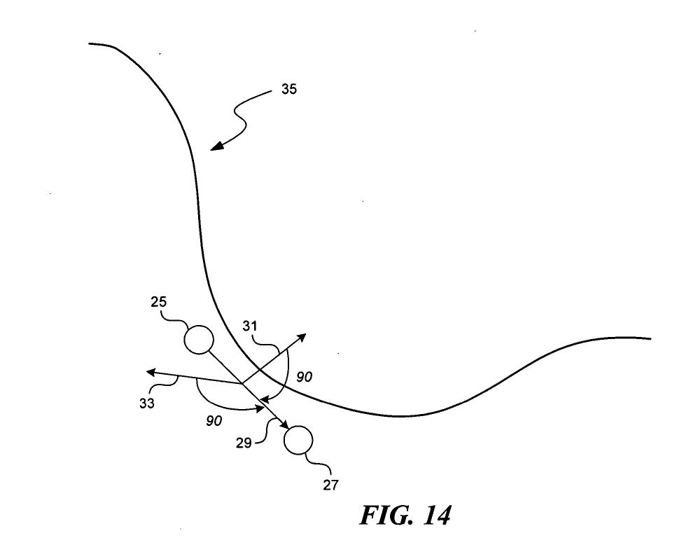

As used herein a body fixed coordinate system refers to a coordinate system where the coordinates of a protein surface are invariant upon rigid rotation or translation of the surface. In general a body fixed coordinate system is may be uniquely defined be selecting three non-collinear points on the surface of a protein Alternatively, a body fixed coordinate system may be define by selecting two points on the surface of the a protein and defining a vector normal to the surface at a third point. There is no inherent limitation on the nature of the coordinate systems that may be employed. One coordinate system, illustrated in FIG. 14, that may be conveniently for building geometric hashing tables is defined by: 1) a first vector 29 connecting two points 25, 27, on the surface of the functional site 35; 2) a second vector 31 normal to both the first vector 29 and the surface of the functional site 35 pointing towards the solvent; and 3) a third vector 33 normal to the first and second vectors 29, 31. If the first basis vector is defined by reference to two residues and if a protein comprises n residues there are n(n−1)/2 possible coordinate systems that may be generated by varying the two residues that define the coordinate system. An infinite number of coordinate system may be determined if points between residues are considered. In order to limit the number of coordinate systems that are determined for each pair of sites, an alternative method selects those residue pairs that are within a threshold distance of each other. Suitable thresholds may range from 10-20 Angstroms. As one skilled in the art will appreciate, while it is generally preferable to determine as many coordinate system as is computationally practicable, at the cost of not sampling rotational/translational transformations of a protein surface, and therefore the accuracy of the claimed methods, less than all of the potential coordinate systems that may be defined on the surface of a protein may be used.

Two hash table entries selected from the first and second hashing tables are considered identical if: i) if the coordinate systems are identical within a coordinate threshold range; and ii) if the coordinates of the single residue are identical within a coordinate threshold range 165. The coordinate system threshold range refers to range of coordinates about a first coordinate that a second coordinate is considered identical. A preferred coordinate threshold range is 0.5-5 Angstroms with 2-4 Angstroms more preferred. Thus, if a first coordinate is x, y, z, and the coordinate threshold range is 2 Angstroms, all coordinates within x²+y²+z²=4 would be considered identical to x, y, z.

The second step in the method illustrated in FIG. 12 166, may sufficiently employ the methods for representing a functional site with a graph detailed in the subsection entitled Graph Based Methods within the section entitled Methods for Determining a Surface that Represents the Physiochemical Topography of a Functional Site-3, 5.

The third step 167, of the method illustrated in FIG. 12 may sufficiently use the methods according to the invention for determining a physiochemical similarity score, S, detailed in the section entitled Methods for Determining a Physiochemical Similarity Score, S-9. While the surface comparison methods according to this aspect of the uses a physiochemical similarity score, S, the method may alternatively also score the similarity of two functional sites using the chemical similarity score, E.

Application of the Protein Similarity Scoring Methods

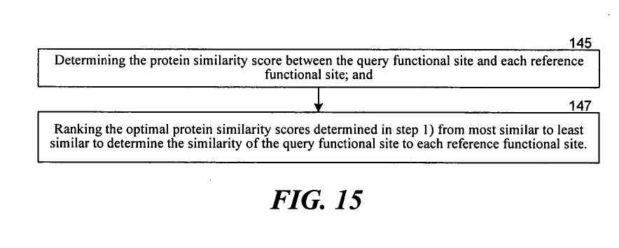

The method detailed in FIG. 1 may be applied to rank the similarity of a query functional site to a plurality of reference functional sites. As used herein a query functional site refers to the functional site that being compared to the reference functional sites using the methods according to the invention. Accordingly, another aspect of the invention, illustrated in FIG. 15, is a method for determining the similarity of a query functional site to a plurality of reference functional sites comprising the steps of: 1) determining the protein similarity score between the query functional site and each reference functional site 145 using the protein similarity scoring methods illustrated in FIGS. 1, 11, or 12; and 2) ranking the protein similarity scores determined in step 1) from most similar to least similar to determine the similarity of the query functional site to each reference functional site 147.

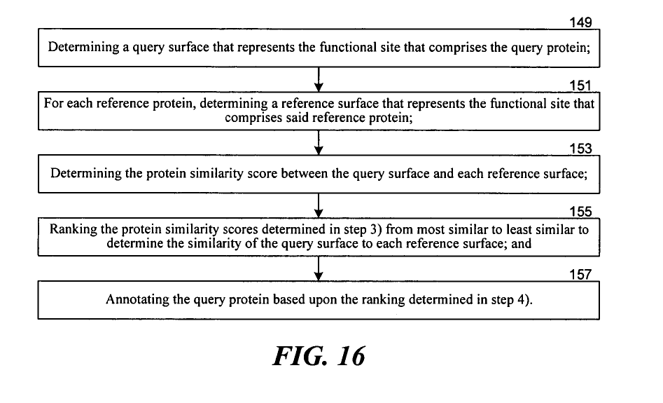

The methods illustrated in FIGS. 1 and 15 may also be used for annotating a query protein comprising a functional site with a database of reference proteins that each comprises a characterized functional site. Accordingly, a still further aspect of the invention, illustrated in FIG. 16, is a method for annotating a query protein comprising a functional site with a database of reference proteins that each comprise a characterized functional site comprising the steps of: 1) determining a query surface that represents the functional site that comprises the query protein 149; 2) for each reference protein, determining a reference surface that represents the functional site of said reference protein 151; 3) determining the protein similarity score between the query surface and each reference surface in the database 153; 4) ranking the protein similarity scores determined in step 3) to determine the similarity of the query surface to each reference surface 155; and 5) annotating the query protein based upon the ranking of optimal protein similarity scores determined in step 4) 157. As used herein, a query surface refers to the mathematical surface used to represent the functional site on a query protein. As used herein, a query protein refers to the protein that is being scored with methods according to the invention to determine its similarity to one or more reference proteins. As used herein, a reference surface refers to the mathematical surface used to represent the functional site on a reference protein.

In order to illustrate the application of the method shown in FIG. 16, consider the case of a query protein comprising a functional site of unknown function and a database comprising ten reference proteins that each comprises a well-characterized functional site of know biological function. A first step determines a surface that represents the functional site that comprises the query protein using the methods illustrated in FIG. 2. A next step, determines ten reference surfaces using Methods illustrated in FIG. 2. A next step, determines the protein similarity score between the query surface and each reference surface using the methods of FIGS. 1, 11, or 12. A next step, ranks the ten reference surfaces from most to least similar (to the query surface) based upon their corresponding protein similarity scores. A next step identifies one or more of the most similar reference surfaces to the query surface for the purposes of annotating the functional site corresponding to the query surface. A final step annotates the functional site on the query protein by assigning to it one or more characteristics, for example, the biological function, from the most similar functional sites found on the reference proteins. The functional annotation may reflect a direct assignment of function from the most similar reference protein or a composite annotation reflecting characteristics from several of the most similar reference proteins.

Systems According to the Invention

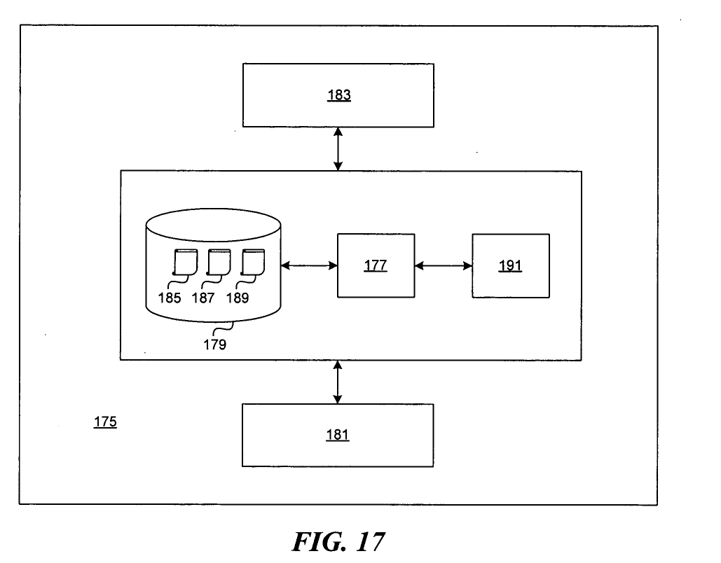

In general, as is shown in FIG. 17, a system according to the invention 175 comprises a processor 177, a memory 179, optionally, an input device 181, optionally, an output device 183, programming for an operating system 185, programming for the methods according to the invention 187, and optionally, programming for storing and retrieving a plurality of functional site structures 189. The systems according to the invention may, optionally, also comprise a device for networking to another device 191.

A processor 177, as used herein, may include one or more microprocessor(s), field programmable logic array(s), or one or more application specific integrated circuit(s). Exemplary processors include, but are not limited to, Intel Corp.'s Pentium series processor (Santa Clara, Calif.), Motorola Corp.'s PowerPC processors (Schaumberg, Ill.), MIPS Technologies Inc.'s MIPs processors (Mountain View, Calif.), or Xilinx Inc.'s Vertex series of field programmable logic arrays (San Jose, Calif.).

A memory 179, as used herein, is any electronic, magnetic or optical based media for storing, reading and writing digital information or a combination of such media. Exemplary types of memory include, but are not limited to, random access memory, electronically programmable read only memory, flash memory, magnetic based disk and tape drives, and optical based disk drives. The memory stores: 1) programming for the methods according to the invention; 2) programming for an operating system and 3) programming for storing and retrieving a plurality of functional site structures.

An input device 181, as used herein, is any device that accepts and processes information from a user. Exemplary devices include, but are not limited to, a keyboard and a pointing device such as a mouse, trackball, joystick or a touch screen/tablet, a microphone with corresponding speech recognition software, any removable optical, magnetic or electronic media based drive, such as a floppy disk drive, a removable hard disk drive, a Compact Disk/Digital Video Disk drive, a flash memory reader or some combination thereof.

An output device 183, as used herein, is any device that processes and outputs information to a user. Exemplary devices include, but are not limited to, visual displays, speakers and or printers. A visual display may be based upon any technology known in the art for processing and presenting a visual image to a user, including cathode ray tube based monitors/projectors, plasma based monitors, liquid crystal display based monitors, digital micro-mirror device based projectors, or light-valve based projectors.

Programming for an operating system 185, as used herein, refers to any machine code, executed by the processor, 177, for controlling and managing the data flow between the processor 177, the memory, the input device 181, the output device 183, and any networking devices 191. In addition to managing data flow among the hardware components that comprise a computer system, an operating system also provides, scheduling, input-output control, file and data management, memory management, and communication control and related services, all in accordance with known methodologies. Exemplary operating systems include, but are not limited to, Microsoft Corp's Windows and NT (Redmond, Wash.), Sun Microsystem Inc.'s Solaris Operating System (Palo Alto, Calif.), Red Hat Corp.'s version of Linux (Durham, N.C.) and Palm Corp.'s PALM OS (Milpitas, Calif.).

Programming for storing and retrieving a plurality functional site structures 189, as used herein, refers to machine code, that when executed by the processor, allows for the storing, retrieving, and organizing of functional site structures or protein structures with annotated functional sites. Exemplary software includes, but is not limited to, relational and object oriented databases such as Oracle Corp.'s 9i (Redwood City, Calif.), International Business Machine, Inc.'s, DB2 (Armonk, N.Y.), Microsoft Corp.'s Access (Redmond, Wash.) and Versant Corp.'s (Freemont, Calif.) Versant Developer Suite 6.0. If functional site structures are stored as flat files, programming for storing and retrieving a plurality of structures and sequences includes programming for operating systems.

Programming for the methods according to the invention 187, as used herein, refers to machine code, that when executed by the processor, performs the methods according to the invention.

A networking device 191 as used herein refers to a device that comprises the hardware and software to allow a system according to the invention to electronically communicate either directly or indirectly to a network server, network switch/router, personal computer, terminal, or other communications device over a distributed communications network. Exemplary networking schemes may be based on packet over any media and include but are not limited to, Ethernet 10/100/1000, IEEE 802.11x, SONET, ATM, IP, MPLS, IEEE 1394, xDSL, Bluetooth, or any other ANSI approved standard.

It will be appreciated by one skilled in the art that the programming for an operating system 185, the programming for storing and retrieving a plurality of functional site structures 189, and the programming for the methods according to the invention 187 may be loaded on to a system according to the invention through either the input device 181, a networking device 191, or a combination of both.

Systems according to the invention may be based upon personal computers (“PCs”) or network servers programmed to perform the methods according to the invention. A suitable server and hardware configuration is an enterprise class Pentium based server, comprising an operating system such as Microsoft's NT, Sun Microsystems' Solaris or Red Hat's version of Linux with 1 GB random access memory, 100 GB storage, either a line area network communications card, such as a 10/100 Ethernet card or a high speed Internet connection, such as a T1/E1 line, optionally, an enterprise database and programming for the methods according to the invention. The storage and memory requirements listed above are not intended to represent minimum hardware configurations, rather they represent a typical server system which may readily purchased from vendors at the time of filing. Such servers may be readily purchased from Dell, Inc. (Austin, Tex.), or Hewlett-Packard, Inc., (Palo Alto, Calif.) with all the features except for the enterprise database and the programming for the methods according to the invention. Enterprise class databases may be purchased from Oracle Corp. or International Business Machines, Inc. It will be appreciated by one skilled in the art that one or more servers may be networked together. Accordingly, the programming for the methods according the invention and an enterprise database for storing and retrieving a plurality of functional site structures may be stored on physically separate servers in communication with each other.

A suitable desktop PC and hardware configuration is a Pentium based desktop computer comprising at least 128 MB of random access memory, 10 GB of storage, a Windows or Linux based operating system, optionally, either a line area network communications card, such as a 10/100 Ethernet card or a high speed Internet connection, such as a T1/E1 line, optionally, a TCP/IP web browser, such as Microsoft's Internet Explorer or the Mozilla Web Browser, optionally, a database such as Microsoft's Access and programming for the methods according to the invention. Once again, the exemplary storage and memory requirement are only intended to represent PC configurations which are readily available from vendors at the time of filing. They are not intended to represent minimum configurations. Such PCs may be readily purchased from Dell, Inc. or Hewlett-Packard, Inc., (Palo Alto, Calif.) with all the features except for the programming for the methods according to the invention.

Although the invention has been described with reference to preferred embodiments and specific examples, it will be readily appreciated by those skilled in the art that many modifications and adaptations of the invention are possible without deviating from the spirit and scope of the invention. Thus, it is to be clearly understood that this description is made only by way of example and not as a limitation on the scope of the invention as claimed below. All references herein are hereby incorporated by reference.

illustrates a chemical similarity scoring scheme based upon the amino acid substitution scoring
matrix of Miyata, A., Miyazawa, S., and Yasunaga, T., Two Types of Amino Acid Substitutions in
Protein Evolution, J. Mol. Evol. 12, 219-236 (1979).

| | A | R | N | D | C | Q | E | G | H | I | L | K | M | F | P | S | T | W | Y | Z |
|---|---|---|---|---|---|---|---|---|---|---|---|---|---|---|---|---|---|---|---|---|
| A | 6 | | | | | | | | | | | | | | | | | | | |
| R | 3.1 | 6 | | | | | | | | | | | | | | | | | | |
| N | 4.2 | 4 | 6 | | | | | | | | | | | | | | | | | |
| D | 3.6 | 3.7 | 5.3 | 6 | | | | | | | | | | | | | | | | |
| C | 4.6 | 2.9 | 3.2 | 2.5 | 6 | | | | | | | | | | | | | | | |
| Q | 4.1 | 4.9 | 5 | 4.5 | 3.5 | 6 | | | | | | | | | | | | | | |
| E | 3.5 | 4.5 | 5.1 | 5.1 | 2.7 | 5.2 | 6 | | | | | | | | | | | | | |
| G | 5.1 | 2.4 | 4 | 3.6 | 3.8 | 3.5 | 3.2 | 6 | | | | | | | | | | | | |
| H | 3.8 | 5.2 | 4.7 | 4.3 | 3.4 | 5.7 | 5 | 3.2 | 6 | | | | | | | | | | | |
| I | 3.3 | 3.5 | 2.6 | 2 | 4.4 | 3.4 | 2.6 | 2.4 | 3.5 | 6 | | | | | | | | | | |
| L | 3.2 | 3.4 | 2.5 | 1.9 | 4.3 | 3.3 | 2.5 | 2.3 | 3.4 | 5.9 | 6 | | | | | | | | | |
| K | 3 | 5.6 | 4.2 | 4 | 2.7 | 4.9 | 4.9 | 2.5 | 5.2 | 3.2 | 3 | 6 | | | | | | | | |
| M | 3.6 | 3.7 | 3 | 2.3 | 4.5 | 3.7 | 2.9 | 2.7 | 3.8 | 5.7 | 5.6 | 3.4 | 6 | | | | | | | |
| F | 2.8 | 3.5 | 2.3 | 1.7 | 3.8 | 3.2 | 2.4 | 1.9 | 3.4 | 5.3 | 5.4 | 3.2 | 5.2 | 6 | | | | | | |
| P | 5.94 | 3.1 | 4.2 | 3.6 | 4.7 | 4.1 | 3.5 | 5 | 3.8 | 3.4 | 3.3 | 3.1 | 3.6 | 2.8 | 6 | | | | | |
| S | 5.5 | 3.3 | 4.7 | 4.1 | 4.2 | 4.3 | 4 | 5.1 | 4.1 | 3 | 3 | 3.3 | 3.3 | 2.6 | 5.4 | 6 | | | | |
| T | 5.1 | 4 | 4.6 | 4 | 4.5 | 4.9 | 4.2 | 4.3 | 4.7 | 3.9 | 3.7 | 3.9 | 4.1 | 3.4 | 5.1 | 5.1 | 6 | | | |
| W | 1.8 | 3.3 | 1.6 | 1.1 | 2.7 | 2.6 | 2 | 0.9 | 2.8 | 4.3 | 4.3 | 2.9 | 4.1 | 4.9 | 1.8 | 1.6 | 2.5 | 6 | | |
| Y | 2.82 | 4 | 2.6 | 2 | 3.6 | 3.5 | 2.8 | 1.9 | 3.7 | 5.1 | 5.1 | 3.6 | 5.1 | 5.5 | 2.9 | 2.7 | 3.6 | 4.9 | 6 | |
| Z | 4.15 | 3.6 | 3.2 | 2.6 | 5.1 | 3.9 | 3 | 3.2 | 3.9 | 5.2 | 5.1 | 3.3 | 5.4 | 4.6 | 4.2 | 3.8 | 4.6 | 3.5 | 4.5 | 6 |

1. A method for determining a mathematical surface that represents a protein functional site comprising the steps of:
a. determining the positions and identities of the residues that comprise said functional site;
b. determining a set of Delaunay tetrahedrons based upon the positions and identities of all or substantially all of the residues that comprise said functional site;
c. determining the Alpha Shape of said functional site from its Delaunay tetrahedrons;
d. identifying empty, connected Delaunay tetrahedrons based upon the Delaunay tessellation and the Alpha Shape of said functional site;
e. determining a set of fused spheres; wherein each sphere is inscribed by an empty Delaunay tetrahedron identified in step d);
f. locating a pseudocenter within the van der Waal's volume of each residue that comprises said functional site, thereby determining a set of pseudocenters aε{1 . . . a′};
g. determining a set of spheres, wherein each said sphere is centered about a pseudocenter determined in step f);
h. determining the subsurface on the set of fused spheres determined in step e) that is subtended by the set of spheres determined in step g), thereby determining a set of connected spherical caps, {σₐ|a=1 . . . a′}, where σₐ is the spherical cap associated with a pseudocenter a; and
i. identifying said mathematical surface with the set of connected spherical caps {σₐ|a=1 . . . a′}.

2. The method of claim 1 wherein each said sphere determined in step g) is centered about a pseudocenter and has a radius of 2-4 Angstroms.

3. The method of claim 1 wherein each said sphere determined in step g) is centered about a pseudocenter and has a radius of 2.9-3.1 Angstroms.

4. A method for determining a mathematical surface that represents a protein functional site comprising the steps of:
a. determining the identity and position of the residues that comprise said functional site;
b. determining a set of Delaunay tetrahedrons based upon the positions and identities of all or substantially all of the residues that comprise said functional site;
c. determining the Alpha Shape of said functional site from its Delaunay tetrahedrons;
d. identifying empty, connected Delaunay tetrahedrons based upon the Delaunay tessellation and the Alpha Shape of said functional site;
e. determining a set of fused spheres; wherein each sphere is inscribed by an empty Delaunay tetrahedron identified in step d);
f. locating and determining the types of one ore more pseudocenters based upon the positions and identities of the residues that comprises said functional site according to Table 1, thereby determining a set of pseudocenters aε{1 . . . a′};
g. determining a set of spheres, wherein each said sphere is centered about a pseudocenter determined in step f);
h. determining the subsurface on the set of fused spheres determined in step e) that is subtended by the set of spheres determined in step g), thereby determining a set of connected spherical caps, {σₐ|a=1 . . . a′}, where σₐ is the spherical cap associated with a pseudocenter a of a type determined in step f); and
i. identifying said mathematical surface with the set of connected spherical caps {σₐ|a=1 . . . a′}.

5. The method of claim 4 wherein each said sphere determined in step g) is centered about a pseudocenter and has a radius of 2-4 Angstroms.

6. The method of claim 4 wherein each said sphere determined in step g) is centered about a pseudocenter and has a radius of 2.9-3.1 Angstroms.

7. A method for determining a mathematical surface that represents a protein functional site comprising the steps of:
a. determining the positions and identities of the residues that comprise said functional site;
b. determining a set of Delaunay tetrahedrons based upon the positions and identities of all or substantially all of the residues that comprise said functional site;
c. determining the Alpha Shape of said functional site from its Delaunay tetrahedrons;
d. identifying empty, connected Delaunay tetrahedrons based upon the Delaunay tessellation and the Alpha Shape of said functional site;
e. determining a set of fused spheres; wherein each sphere is inscribed by an empty Delaunay tetrahedron identified in step d);
f. locating a pseudocenter at the center-of-mass of the side chain of each residue that comprises said functional site, thereby determining a set of pseudocenters aε{1 . . . a′};
g. assigning to each pseudocenter an identification of its corresponding residue's identity, thereby assigning a pseudocenter type to said pseudocenter
h. determining a set of spheres, wherein each said sphere is centered about a pseudocenter determined in step f);
i. determining the subsurface on the set of fused spheres determined in step e) that is subtended by the set of spheres determined in step h), thereby determining a set of connected spherical caps, {σₐ|a=1 . . . a′}, where σₐ is the spherical cap associated with a pseudocenter a of a type determined in step g); and
identifying said mathematical surface with the set of connected spherical caps {σₐ|a=1 . . . a′}.

8. The method of claim 7 wherein each said sphere determined in step h) is centered about a pseudocenter and has a radius of 2-4 Angstroms.

9. The method of claim 7 wherein each said sphere determined in step h) is centered about a pseudocenter and has a radius of 2.9-3.1 Angstroms.

10. A method comprising the steps of:
a. selecting first and second functional sites;
b. determining the identities and positions of the residues that comprise the first and second functional sites, thereby determining first and second functional site structures;
c. determining a first surface of the form Σ₁={σₐ|a=1 . . . a′}, wherein σₐ is a spherical cap corresponding to pseudocenter a, based upon the first functional site structure using the method of claim 6;
d. determining a second surface of the form Σ₂={σ_{b}|b=1 . . . b′}, wherein σ_{b} is a spherical cap corresponding to pseudocenter b, based upon the second functional site structure using the method of claim 6;
e. determining the distance between each pseudocenter a corresponding to a spherical cap in the first set of spherical caps and each pseudocenter b corresponding to a spherical cap in the second set of spherical caps;
f. selecting a first spherical cap σₐ₌₁ from the first set of spherical caps and a second spherical cap σ_{b=1} from the second set of spherical caps that corresponds to a pseudocenter b=1 that is closest to the pseudocenter a=1 corresponding to the first spherical cap;
g. defining a normal unit vector at the midpoint of each spherical cap;
h. rotating the first spherical cap σₐ₌₁ such that the normal vector to the first spherical cap is collinear to the normal vector to second spherical cap σ_{b-1};
i. determining a volume score, Vₐ₌₁^{R}, that represents volume of rotation produced by the rotation of the first spherical cap; j. translating the first spherical cap until its normal vector is coincident with the normal vector to the second spherical cap; k. determining a volume score, Vₐ₌₁^{T}, that represents the volume of translation produced by translating the first spherical cap;
l. determining a volume score, V_{a=1,b=1}^{E}that represent the volume of exclusion between the two spherical caps;
m. determining V_{a=1,b=1} by summing the quantities determined in steps i), k) and l);
n. determining a spherical cap chemical similarity score E_{a=1,b=1} between the first and second spherical caps σₐ₌₁,σ_{b=1}, based upon their corresponding pseudocenter types a,b and according to Table 3;
o. determining a physiochemical similarity score of the form S_{a=1,b=1}=E_{a=1,b=1}ƒ(V_{a=1,b=1}), wherein ƒ(V_{a=1,b=1}) is a monotonically increasing function of the volume V_{a,b} between the spherical caps σₐ and σ_{b};
p. repeating steps f) through o) until S_{a,b} has been determined for a plurality of spherical cap pairs that are each formed by selecting one spherical cap σₐ from the first set of spherical caps Σ₁ and a second spherical cap σ_{b} from the second set of spherical caps Σ₂ whose corresponding pseudocenter b is closest to the pseudocenter a corresponding to the spherical cap σₐ that is selected from the first set of spherical caps Σ₁; and
q. determining a physiochemical similarity score S of the form
$$S = {\sum }_{{a = 1}_{b = 1} ,}^{{a}^{′} , {b}^{′}} {S}_{a , b} .$$

11. A method comprising the steps of:
a. selecting first and second functional sites;
b. determining the identities and positions of the residues that comprise the first and functional sites, thereby determining first and second functional site structures;
c. determining a first surface of the form Σ₁={σₐ|a=1 . . . a′}, wherein σₐ is a spherical cap corresponding to pseudocenter a, based upon the first functional site structure using the method of claim 6;
d. determining a second surface of the form Σ₂={σ_{b}|b=1 . . . b′}, wherein σ_{b} is a spherical cap corresponding to pseudocenter b, based upon the second functional site structure using the method of claim 6;
e. determining the distance between each pseudocenter a corresponding to a spherical cap in the first set of spherical caps and each pseudocenter b corresponding to a spherical cap in the second set of spherical caps;
f. selecting a first spherical cap σₐ₌₁ from the first set of spherical caps and a second spherical cap σ_{b=1} from the second set of spherical caps that corresponds to a pseudocenter b=1 that is closest to the pseudocenter a=1 corresponding to the first spherical cap;
g. defining a normal unit vector at the midpoint of each spherical cap;
h. rotating the first spherical cap σₐ₌₁ such that the normal vector to the first spherical cap is collinear to the normal vector to second spherical cap σ_{b=}1;
i. determining a volume score, Vₐ₌₁^{R}, that represents volume of rotation produced by the rotation of the first spherical cap;
j. translating the first spherical cap until its normal vector is coincident with the normal vector to the second spherical cap;
k. determining a volume score, Vₐ₌₁^{T}, that represents the volume of translation produced by translating the first spherical cap;
l. determining a volume score, V_{a=1,b=1}^{E}, that represent the volume of exclusion between the two spherical caps;
m. determining V_{a=1,b=1} by summing the quantities determined in steps i), k) and l);
n. determining a spherical cap chemical similarity score E_{a=1,b=1} between the first and second spherical caps σₐ₌₁, σ_{b=1}, based upon their corresponding pseudocenter types a,b and according to Table 3;
o determining a physiochemical similarity score of the form S_{a=1,b=1}=ƒ(E_{a=1,b=1})V_{a=1,b=1}, wherein ƒ(E_{a=1,b=1}) is a monotonically decreasing function of the chemical similarity E_{a,b} between the spherical caps σₐ and σ_{b};
p. repeating steps f) through o) until S_{a,b} has been determined for a plurality of spherical cap pairs that are each formed by selecting one spherical cap σₐ from the first set of spherical caps Σ₁ and a second spherical cap σ_{b} from the second set of spherical caps Σ₂ whose corresponding pseudocenter b is closest to the pseudocenter a corresponding to the spherical cap σₐ that is selected from the first set of spherical caps Σ₁; and
q. determining a physiochemical similarity score S of the form
$$S = {\sum }_{\begin{matrix}a = 1 , \\ b = 1\end{matrix}}^{{a}^{′} , {b}^{′}} {S}_{a , b} .$$

12. A method comprising the steps of:
a. selecting first and second functional sites;
b. determining the identities and positions of the residues that comprise the first and second functional sites, thereby determining first and second functional site structures;
c. determining a first surface of the form Σ₁={σₐ|a=1 . . . a′}, wherein σₐ is a spherical cap corresponding to pseudocenter a, based upon the first functional site structure using the method of claim 9;
d. determining a second surface of the form σ₂={σ_{b}|b=1 . . . b′}, wherein σ_{b} is a spherical cap corresponding to pseudocenter b, based upon the second functional site structure using the method of claim 9;
e. determining the distance between each pseudocenter a corresponding to a spherical cap in the first set of spherical caps and each pseudocenter b corresponding to a spherical cap in the second set of spherical caps;
f. selecting a first spherical cap σₐ₌₁ from the first set of spherical caps and a second spherical cap σ_{b=1} from the second set of spherical caps that corresponds to a pseudocenter b=1 that is closest to the pseudocenter a=1 corresponding to the first spherical cap;
g. defining a normal unit vector at the midpoint of each spherical cap;
h. rotating the first spherical cap σₐ₌₁ such that the normal vector to the first spherical cap is collinear to the normal vector to second spherical cap σ_{b=1};
i. determining a volume score, Vₐ₌₁^{R}, that represents volume of rotation produced by the rotation of the first spherical cap;
j. translating the first spherical cap until its normal vector is coincident with the normal vector to the second spherical cap;
k. determining a volume score, Vₐ₌₁^{T}, that represents the volume of translation produced by translating the first spherical cap;
l. determining a volume score, V_{a=1,b=1}^{E}, that represent the volume of exclusion between the two spherical caps;
m. determining V_{a=1,b=1} by summing the quantities determined in steps i), k) and l);
n. determining a spherical cap chemical similarity score E_{a=1,b=1} between the first and second spherical caps σₐ₌₁, σ_{b=1}, based upon their corresponding pseudocenter types a,b and according to Table 2;
o. determining a physiochemical similarity score of the form S_{a=1,b=1}=E_{a=1,b=1}ƒ(V_{a=1,b=1}), wherein ƒ(V_{a=1,b=1}) is a monotonically increasing function of the volume V_{a,b} between the spherical caps σₐ and σ_{b};
p. repeating steps f) through o) until S_{a,b} has been determined for a plurality of spherical cap pairs that are each formed by selecting one spherical cap σₐ from the first set of spherical caps Σ₁ and a second spherical cap σ_{b} from the second set of spherical caps Σ₂ whose corresponding pseudocenter b is closest to the pseudocenter a corresponding to the spherical cap σₐ that is selected from the first set of spherical caps Σ₁; and
q. determining a physiochemical similarity score S of the form
$$S = {\sum }_{\begin{matrix}a = 1 , \\ b = 1\end{matrix}}^{{a}^{′} , {b}^{′}} {S}_{a , b} .$$

13. A method comprising the steps of:
a. selecting first and second functional sites;
b. determining the identities and positions of the residues that comprise the first and functional sites, thereby determining first and second functional site structures;
c. determining a first surface of the form Σ₁={σₐ|a=1 . . . a′}, wherein σₐ is a spherical cap corresponding to pseudocenter a, based upon the first functional site structure using the method of claim 9;
d. determining a second surface of the form Σ₂={σ_{b}|b=1 . . . b′}, wherein σ_{b} is a spherical cap corresponding to pseudocenter b, based upon the second functional site structure using the method of claim 9;
e. determining the distance between each pseudocenter a corresponding to a spherical cap in the first set of spherical caps and each pseudocenter b corresponding to a spherical cap in the second set of spherical caps;
f. selecting a first spherical cap σₐ₌₁ from the first set of spherical caps and a second spherical cap σ_{b=1} from the second set of spherical caps that corresponds to a pseudocenter b=1 that is closest to the pseudocenter a=1 corresponding to the first spherical cap;
g. defining a normal unit vector at the midpoint of each spherical cap;
h. rotating the first spherical cap σₐ₌1 such that the normal vector to the first spherical cap is collinear to the normal vector to second spherical cap σ_{b=1};
i. determining a volume score, Vₐ₌₁^{R}, that represents volume of rotation produced by the rotation of the first spherical cap;
j. translating the first spherical cap until its normal vector is coincident with the normal vector to the second spherical cap;
k. determining a volume score, Vₐ₌₁^{T}, that represents the volume of translation produced by translating the first spherical cap;
l. determining a volume score, V_{a=1,b=1}^{E}, that represent the volume of exclusion between the two spherical caps;
m. determining V_{a=1,b=1} by summing the quantities determined in steps i), k) and l);
n. determining a spherical cap chemical similarity score E_{a=1,b=1} between the first and second spherical caps σₐ₌₁, σ_{b=1} based upon their corresponding pseudocenter types a,b and according to Table 2;
o determining a physiochemical similarity score of the form S_{a=1,b=1}=ƒ(E_{a=1,b=1})V_{a=1,b=1}, wherein ƒ(E_{a=1,b-1}) is a monotonically decreasing function of the chemical similarity E_{a,b} between the spherical caps σₐ and σ_{b};
p. repeating steps f) through o) until S_{a,b} has been determined for a plurality of spherical cap pairs that are each formed by selecting one spherical cap σₐ from the first set of spherical caps Σ₁ and a second spherical cap σ_{b} from the second set of spherical caps Σ₂ whose corresponding pseudocenter b is closest to the pseudocenter a corresponding to the spherical cap σₐ that is selected from the first set of spherical caps Σ₁; and
q. determining a physiochemical similarity score S of the form
$$S = {\sum }_{\begin{matrix}a = 1 , \\ b = 1\end{matrix}}^{{a}^{′} , {b}^{′}} {S}_{a , b} .$$

14. A method comprising the steps of:
a. selecting a first and second functional site;
b. determining the positions and identities of the residues that comprise the first and second functional sites, thereby determining first and second functional site structures;
c. determining a first physiochemical similarity score S using steps c)-q) in the method of claim 12;
d. rigidly transforming said first and second structures thereby determining a second relative orientation;
e. determining a first physiochemical similarity score S using steps c)-q) in the method of claim 12 based upon the second relative orientation of the two surfaces determined in step d);
f. repeating steps d) and e) until a plurality of physiochemical similarity scores corresponding to a plurality of orientations between the two structures have been determined;
g. ranking the physiochemical similarity scores determined in step f); and
h. identifying a protein similarity score with maximum physiochemical similarity score determined in step g).

15. A method comprising the steps of:
a. selecting a first and second functional site;
b. determining the identities and positions of the residues that comprise the first and second functional sites, thereby determining first and second functional site structures;
c. representing the first and second functional site structures with respectively first and second graphs, said graphs each comprising nodes and edges wherein the nodes correspond to residues of each functional site and the edges correspond to the distance between the residues;
d. determining the maximum common subgraph of the first graph and the second graph;
e. identifying the residue pairs and their positions in the first and second functional sites corresponding to the node pairs in the maximum common subgraph;
f. rigidly transforming one of the two functional site structures to overlay the two structures in a relative orientation corresponds to the maximum common subgraph based upon residue pairs and their positions identified in the maximum common subgraph, thereby determining the optimal overlay of the first and second functional site structures;
g. determining the physiochemical similarity score S using steps c)-q) of the method of claim 12 based upon the optimally overlayed functional site structures determined in step f); and
h. identifying a protein similarity score with the physiochemical similarity score S that was determined in step g).

16. A method comprising the steps of:
a. selecting first and second functional sites
b. determining the identities and positions of the residues that comprise the first and second functional sites, thereby determining first and second functional site structures;
c. using geometric hashing to determine one or more relative orientations between the two functional site structures such that in each such relative orientation, at least one residue from the first functional site is coincidental in position to at least one residue from the second functional site;
d. for each such relative orientation of the orientations of the functional site structures determined in step c) determining a physiochemical similarity score S using steps c)-q) in the method of claim 12; and
e. identifying the protein similarity score with the maximum physiochemical similarity score determined in step d).

17. A method for determining similarity of a query functional site to a plurality of reference functional sites comprising the steps of:
a. using step b)-h) of the method of claim 14 to determine a protein similarity score between said query functional site and each said reference functional site; and
b. ranking said protein similarity scores determined in step a) to determine the similarity of the query functional site to each reference functional site.

18. A computer system comprising:
a. an input device;
b. an output device;
c. a processor;
d. a memory;
e. programming for an operating system; and
f. programming for the method of claim 9.

19. A computer system comprising:
a. an input device;
b. an output device;
c. a processor;
d. a memory;
e. programming for an operating system; and
f. programming for the method of claim 10.

20. A computer system comprising:
a. an input device;
b. an output device;
c. a processor;
d. a memory;
e. programming for an operating system;
f. programming for storing and retrieving a plurality of protein structures; and
g. programming for the method of claim 15.
